(19) United States
(12) Patent Application Publication
(10) Pub. No.: US 2016/0029954 A1
(43) Pub. Date: Feb. 4, 2016

(54) EXERCISE ANALYSIS APPARATUS, EXERCISE ANALYSIS SYSTEM, EXERCISE ANALYSIS METHOD, AND EXERCISE ANALYSIS PROGRAM

(71) Applicant: Seiko Epson Corporation, Tokyo (JP)

(72) Inventors: Akinobu Sato, Fujimi-machi (JP); Shuji Uchida, Shiojiri-shi (JP); Shunichi Mizuochi, Matsumoto-shi (JP)

(21) Appl. No.: 14/811,730

(22) Filed: Jul. 28, 2015

(30) Foreign Application Priority Data
Jul. 31, 2014 (JP) 2014-157205

(51) Int. Cl. A61B 5/00; G01P 15/02; G01P 3/00

(52) U.S. Cl. CPC A61B 5/681; G01P 3/00; G01P 15/02; A61B 5/6804

(57) ABSTRACT

An exercise analysis apparatus includes: an acquisition unit which acquires a detection result from a sensor (inertial measurement unit) which detects information relating to an exercise performed by a user equipped with the sensor; and a right and left determination unit which performs a determination process of determining whether a period is a first period including a right foot ground contact period of the user or a second period including a left foot ground contact period of the user, based on the detection result.

### BACKGROUND

1. Technical Field

The present invention relates to an exercise analysis apparatus, an exercise analysis system, an exercise analysis method, and an exercise analysis program.

2. Related Art

When walking or running, it is important to step smoothly using appropriate form without any bias in balance to the right or left.

JP-A-2008-73285 discloses a walking and running motion evaluation support apparatus which evaluates walking and running motions based on output of gyro sensors attached to the right and left foot.

In a case where the sensors are attached to the right and left foot as disclosed in JP-A-2008-73285, two sensors are necessary, and accordingly, the cost increases and synchronous processing between the sensors is also necessary. When performing the evaluation in real time, a communication process of performing communication with the sensors attached to each foot is also necessary.

### SUMMARY

An advantage of some aspects of the invention is to provide an exercise analysis apparatus, an exercise analysis system, an exercise analysis method, and an exercise analysis program which can grasp exercise motions of right and left foot with a simple configuration.

The invention can be implemented as the following forms or application examples.

### Application Example 1

An exercise analysis apparatus according to this application example includes: an acquisition unit which acquires a detection result from a sensor which detects information relating to an exercise performed by a user equipped with the sensor; and a right and left determination unit which performs a determination process of determining whether a period is a first period including a right foot ground contact period of the user or a second period including a left foot ground contact period of the user, based on the detection result.

According to this application example, since the determination process can be performed with one sensor, it is possible to realize an exercise analysis apparatus which can grasp exercise motions of right and left foot with a simple configuration.

### Application Example 2

In the exercise analysis apparatus described above, the sensor may be an inertial sensor which detects at least one of an angle and an angular velocity.

Since the inertial sensor can detect small movements of a user equipped with the sensor, it is possible to realize an exercise analysis apparatus which can precisely grasp exercise motions of right and left foot with one sensor.

### Application Example 3

In the exercise analysis apparatus described above, the right and left determination unit may perform the determination process based on an angular velocity of the user in a yaw direction.

In a walking operation or a running operation, a turning exercise in the yaw direction occurs in the body of a user. Therefore, according to this application example, it is possible to realize an exercise analysis apparatus which can precisely grasp exercise motions of right and left foot with one sensor.

### Application Example 4

In the exercise analysis apparatus described above, the right and left determination unit may perform the determination process based on an acceleration in a direction orthogonal to both of a proceeding direction of the user and a vertical direction.

In a walking operation or a running operation, there is a direction orthogonal to both the proceeding direction of the user and the vertical direction. Therefore, according to this application example, it is possible to realize an exercise analysis apparatus which can precisely grasp exercise motions of right and left foot with one sensor.

### Application Example 5

In the exercise analysis apparatus described above, the right and left determination unit may perform the determination process based on an angle of the user in the yaw direction.

In a walking operation or a running operation, a turning exercise in the yaw direction occurs in the body of a user. Therefore, according to this application example, it is possible to realize an exercise analysis apparatus which can precisely grasp exercise motions of right and left foot with one sensor.

### Application Example 6

The exercise analysis apparatus described above may further include a running detection unit which detects running timing of the user, and the right and left determination unit may perform the determination process based on the running timing.

According to this application example, the determination process can be performed based on the running timing (for example, timing of one step) and the detection result of the sensor, and therefore, it is possible to realize an exercise analysis apparatus which can precisely grasp exercise motions of right and left foot.

### Application Example 7

The exercise analysis apparatus described above may further include a calculation unit which calculates the index relating to the exercise performed by the user, and the calculation unit may calculate the index in the first period and the index in the second period.

### Application Example 8

In the exercise analysis apparatus described above, the index may be at least one of direct below strike, propulsive efficiency, leg turnover, a running pitch, and strike impact.

According to this application example, it is possible to realize an exercise analysis apparatus which can provide a user with the information relating to the right and left balance of the exercise performed by a user.

### Application Example 9

In the exercise analysis apparatus described above, the calculation unit may calculate a difference between the index in the first period and the index in the second period.

According to this application example, it is possible to realize an exercise analysis apparatus which can provide the information relating to the right and left balance of the exercise performed by a user which a user can easily understand.

### Application Example 10

In the exercise analysis apparatus described above, an output unit which outputs difference information which is information relating to the difference may be further included, when the difference is equal to or greater than a reference value.

According to this application example, it is possible to realize an exercise analysis apparatus which can send a notification to a user, when the right and left balance of the exercise performed by a user is not obtained.

### Application Example 11

An exercise analysis system according to this application example includes: the exercise analysis apparatus described above; and a notification apparatus which outputs the difference information output by the exercise analysis apparatus.

According to this application example, it is possible to realize an exercise analysis system which can send a notification to a user, when the right and left balance of the exercise performed by a user is not obtained.

### Application Example 12

An exercise analysis method according to this application example includes: acquiring a detection result from a sensor which detects information relating to an exercise performed by a user equipped with the sensor; and performing a determination process of determining whether a period is a first period including a right foot ground contact period of the user or a second period including a left foot ground contact period of the user, based on the detection result.

According to this application example, the determination process can be performed with one sensor, and therefore, it is possible to realize an exercise analysis method which can grasp exercise motions of right and left foot with a simple configuration.

### Application Example 13

An exercise analysis program according to this application example causes a computer to function as: an acquisition unit which acquires a detection result from a sensor which detects information relating to an exercise performed by a user equipped with the sensor; and a right and left determination unit which performs a determination process of determining whether a period is a first period including a right foot ground contact period of the user or a second period including a left foot ground contact period of the user, based on the detection result.

According to this application example, since the determination process can be performed with one sensor, therefore, it is possible to realize an exercise analysis program which can grasp exercise motions of right and left foot with a simple configuration.

### BRIEF DESCRIPTION OF THE DRAWINGS

The invention will be described with reference to the accompanying drawings, wherein like numbers reference like elements.

FIG. 1 is a diagram showing a configuration example of an exercise analysis system of the embodiment.

FIG. 2 is an explanatory diagram of an outline of the exercise analysis system of the embodiment.

FIG. 3 is a functional block diagram showing a configuration example of an exercise analysis apparatus.

FIG. 4 is a diagram showing a configuration example of a sensing data table.

FIG. 5 is a diagram showing a configuration example of a GPS data table.

FIG. 6 is a diagram showing a configuration example of a geomagnetic data table.

FIG. 7 is a diagram showing a configuration example of a calculation data table.

FIG. 8 is a functional block diagram showing a configuration example of a processing unit of the exercise analysis apparatus.

FIG. 9 is a functional block diagram showing a configuration example of an inertial navigation operation unit.

FIG. 10 is an explanatory diagram of postures of a user at the time of running.

FIG. 11 is an explanatory diagram of a yaw angle at the time when running is performed by a user.

FIG. 12 is a diagram illustrating a first period and a second period.

FIG. 13 is a graph showing an angular velocity in a yaw direction.

FIG. 14 is a graph showing a y axis acceleration.

FIG. 15 is a diagram showing an example of three-axis acceleration at the time when running is performed by a user.

FIG. 16 is a functional block diagram showing a configuration example of an exercise analysis unit.

FIG. 17 is a flowchart showing an example of a procedure of an exercise analysis process performed by a processing unit.

FIG. 18 is a flowchart showing an example of a procedure of an inertial navigation operation process.

FIG. 19 is a flowchart showing an example of a procedure of a running detection process.

FIG. 20 is a flowchart showing an example of a procedure of a determination process.

FIG. 21 is a flowchart showing an example of a procedure of an exercise analysis information generation process (process in S50 of FIG. 17).

FIG. 22 is a functional block diagram showing a configuration example of a notification apparatus.

FIGS. 23A and 23B are diagrams showing an example of information displayed on a display unit of the notification apparatus.

FIG. 24 is a flowchart showing an example of a procedure of a notification process performed by a processing unit.

FIG. 25 is a functional block diagram showing a configuration example of an information analysis apparatus.

FIG. 26 is a flowchart showing an example of a procedure of an analysis process performed by a processing unit.

FIG. 27 is a diagram showing an example of analysis information displayed on a display unit.

### DESCRIPTION OF EXEMPLARY EMBODIMENTS

Hereinafter, preferred embodiments of the invention will be described with reference to the drawings. The drawings used are shown for convenience of description. The embodiments described below do not unjustly limit the content of the invention disclosed in the appended claims. All of the configurations described below are not limited to being compulsory constituent elements of the invention.

### 1. Exercise Analysis System

### 1-1. Configuration of System

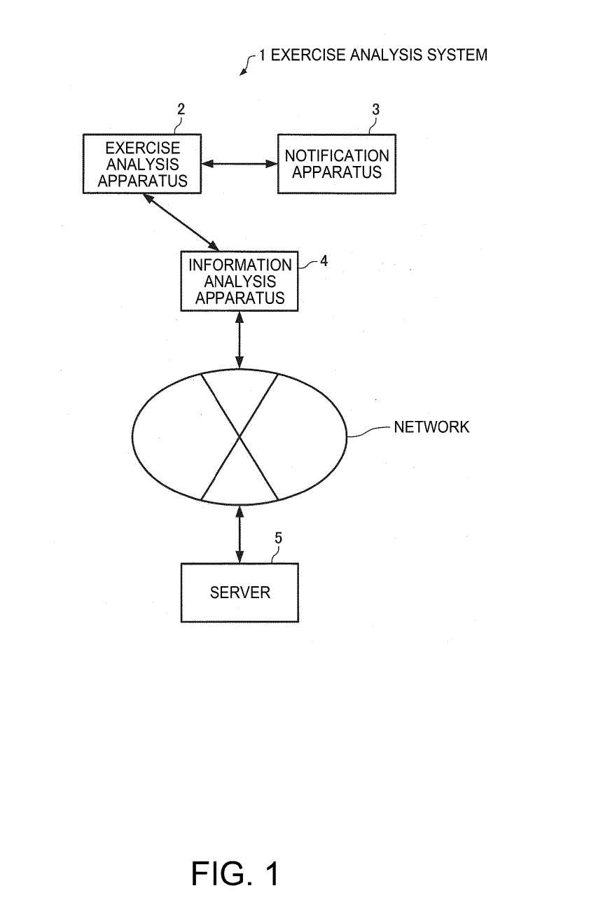

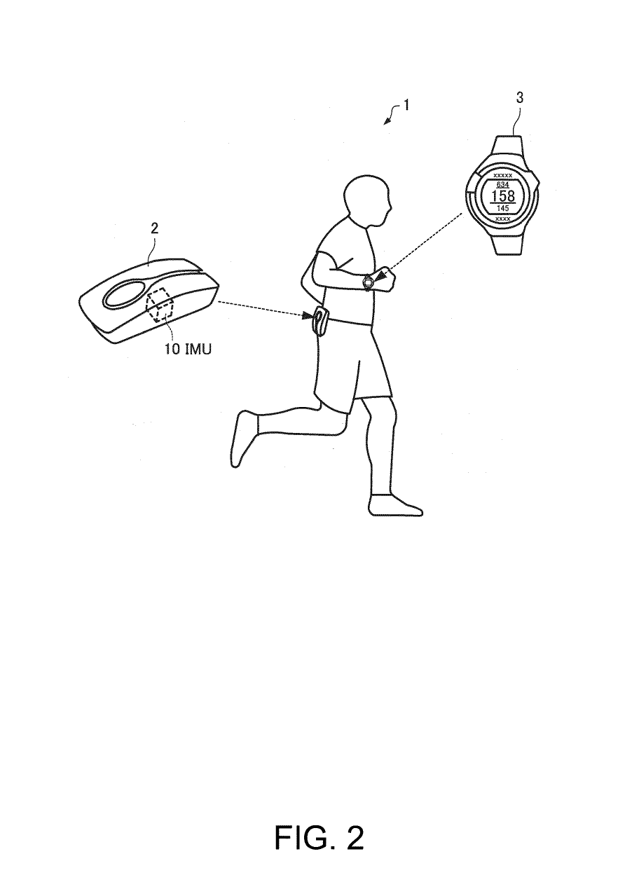

Hereinafter, an exercise analysis system which analyzes an exercise such as running (including walking) performed by a user will be described as an example, but the exercise analysis system of the embodiment can also be applied to an exercise analysis system which analyzes an exercise other than the running, in the same manner. FIG. 1 is a diagram showing a configuration example of an exercise analysis system 1 of the embodiment. As shown in FIG. 1, the exercise analysis system 1 includes an exercise analysis apparatus 2, a notification apparatus 3, and an information analysis apparatus 4. The exercise analysis apparatus 2 is an apparatus which analyzes an exercise performed by a user at the time of running and the notification apparatus 3 is an apparatus which notifies a user of information of a state of the exercise of a user at the time of running or a running result. The information analysis apparatus 4 is an apparatus which analyzes and provides a running result after finishing of the running by a user. In the embodiment, as shown in FIG. 2, the exercise analysis apparatus 2 includes an inertial measurement unit (IMU) 10 embedded therein and is put on the body (for example, left side of the waist, right side of the waist, middle of the waist) of a user so that one detection axis (hereinafter, set as a z axis) of the inertial measurement unit (IMU) 10 substantially coincides with a gravitational acceleration direction (downwards in a vertical direction) in a state where a user stands still. The notification apparatus 3 is a wrist type (watch type) portable information apparatus and is put on a wrist or the like of a user. However, the notification apparatus 3 may be a portable information apparatus such as a head mounted display (HMD) or a smart phone.

A user operates the notification apparatus 3 when starting running to instruct a start of measurement (an inertial navigation operation process and an exercise analysis process which will be described later) performed by the exercise analysis apparatus 2 and operates the notification apparatus 3 when finishing the running to instruct the end of the measurement performed by the exercise analysis apparatus 2. The notification apparatus 3 transmits a command for instructing the start or the end of the measurement to the exercise analysis apparatus 2 according to the operation of a user.

When the command for starting the measurement is received, the exercise analysis apparatus 2 starts the measurement performed by the inertial measurement unit (IMU) 10, calculates values of various exercise indexes which are indexes relating to a running ability (an example of exercise ability) of a user using the measured results, and generates exercise analysis information including the values of various exercise indexes as information regarding analysis results of the running ability of a user. The exercise analysis apparatus 2 generates information (running output information) output during the running performed by a user using the generated exercise analysis information and transmits the information to the notification apparatus 3. The notification apparatus 3 receives the running output information from the exercise analysis apparatus 2, compares various exercise indexes included in the running output information and reference values set in advance, and notifies a user of suitability of each exercise index mainly using sound or vibration. Accordingly, a user can run while checking the suitability of each exercise index.

When the command for finishing the measurement is received, the exercise analysis apparatus 2 finishes the measurement performed by the inertial measurement unit (IMU) 10, generates information (running result information: running distance and running speed) of a running result of a user, and transmits the information to the notification apparatus 3. The notification apparatus 3 receives the running result information from the exercise analysis apparatus 2 and notifies a user of the information regarding the running result as letters or an image. Accordingly, a user can check the information regarding the running result immediately after finishing running.

The data communication between the exercise analysis apparatus 2 and the notification apparatus 3 may be wireless communication or wired communication.

As shown in FIG. 1, in the embodiment, the exercise analysis system 1 includes a server 5 which is connected to a network such as the Internet or a local area network (LAN). The information analysis apparatus 4 is, for example, an information apparatus such as a personal computer or a smart phone and can perform data communication with the server 5 through a network. The information analysis apparatus 4 acquires the exercise analysis information relating to the previous running performed by a user from the exercise analysis apparatus 2 and transmits the exercise analysis information to the server 5 through the network. However, an apparatus other than the information analysis apparatus 4 may acquire the exercise analysis information from the exercise analysis apparatus 2 and transmit the exercise analysis information to the server 5 or the exercise analysis apparatus 2 may directly transmit the exercise analysis information to the server 5. The server 5 receives this exercise analysis information and stores the exercise analysis information in a database created in a storage unit (not shown). In the embodiment, a plurality of users run with the same or a different exercise analysis apparatus 2 attached to their body and the exercise analysis information relating to each user is stored in a database of the server 5.

The information analysis apparatus 4 acquires the exercise analysis information of a plurality of users from the database of the server 5 through a network, generates analysis information for comparing the running abilities of the plurality of users, and displays the analysis information on a display unit (not shown in FIG. 1). The running ability of a specific user can be compared with that of another user to be relatively evaluated or a reference value of each exercise index can be suitably set using the analysis information displayed on the display unit of the information analysis apparatus 4. When a user sets a reference value of each exercise index, the information analysis apparatus 4 transmits setting information regarding the reference value of each exercise index to the notification apparatus 3.

In the exercise analysis system 1, the exercise analysis apparatus 2, the notification apparatus 3, and the information analysis apparatus 4 may be separately provided, the exercise analysis apparatus 2 and the notification apparatus 3 may be integrally provided and the information analysis apparatus 4 may be separately provided, the notification apparatus 3 and the information analysis apparatus 4 may be integrally provided and the exercise analysis apparatus 2 may be separately provided, the exercise analysis apparatus 2 and the information analysis apparatus 4 may be integrally provided and the notification apparatus 3 may be separately provided, or the exercise analysis apparatus 2, the notification apparatus 3, and the information analysis apparatus 4 may be integrally provided. The exercise analysis apparatus 2, the notification apparatus 3, and the information analysis apparatus 4 may be combined in any form.

### 1-2. Coordinate System

A coordinate system necessary in the following description will be defined.
e frame (Earth Centered Earth Fixed Frame): right-handed three-dimensional orthogonal coordinate having a z axis be parallel to a rotation shaft by setting the center of the earth as the origin
n frame (Navigation Frame): three-dimensional orthogonal coordinate setting an x axis as the north, a y axis as the east, and a z axis as a gravity direction by setting a moving body (user) as the origin
b frame (Body Frame): three-dimensional orthogonal coordinate having a sensor (inertial measurement unit (IMU) 10) as a reference
m frame (Moving Frame): right-handed three-dimensional orthogonal coordinate setting a proceeding direction of a moving body (user) as an x axis by setting the moving body (user) as the origin

### 1-3. Exercise Analysis Apparatus

### 1-3-1. Configuration of Exercise Analysis Apparatus

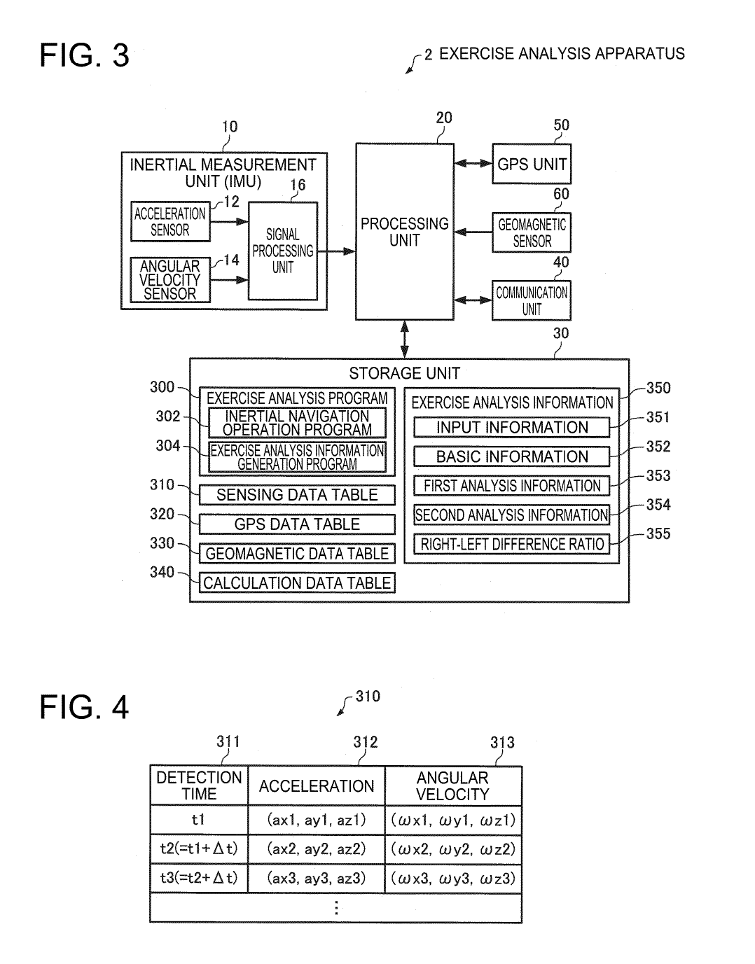

FIG. 3 is a functional block diagram showing a configuration example of the exercise analysis apparatus 2. As shown in FIG. 3, the exercise analysis apparatus 2 includes the inertial measurement unit (IMU) 10, a processing unit 20, a storage unit 30, a communication unit 40, a global positioning system (GPS) unit 50, and a geomagnetic sensor 60. However, in the exercise analysis apparatus 2 of the embodiment, some of these constituent elements may be removed or changed or other constituent elements may be added.

The inertial measurement unit (IMU) 10 (an example of inertial sensor) includes an acceleration sensor 12, an angular velocity sensor 14, and a signal processing unit 16.

The acceleration sensor 12 detects acceleration in each of three axis directions intersecting each other (ideally, orthogonal to each other) and outputs a digital signal (acceleration data) according to magnitude and a direction of the detected three-axis acceleration.

The angular velocity sensor 14 detects angular velocity in each of three axis directions intersecting each other (ideally, orthogonal to each other) and outputs a digital signal (angular velocity data) according to the magnitude and the direction of the measured three-axis angular velocity.

The signal processing unit 16 receives acceleration data and angular velocity data from the acceleration sensor 12 and the angular velocity sensor 14, adds time information to the data, stores the data in a storage unit (not shown), generates sensing data obtained by adding the stored acceleration data, angular velocity data, and time information in a predetermined format, and outputs the sensing data to the processing unit 20.

The acceleration sensor 12 and the angular velocity sensor 14 are ideally mounted so that the three axes coincide with three axes of the sensor coordinate system (b frame) having the inertial measurement unit 10 as a reference, but there is an error of a mounting angle in practice. Therefore, the signal processing unit 16 performs a process of converting the acceleration data and the angular velocity data into data in the sensor coordinate system (b frame), using a correction parameter which is previously calculated according to the mounting angle error. The processing unit 20 which will be described later may perform the conversion process, instead of the signal processing unit 16.

In addition, the signal processing unit 16 performs a temperature correction process of the acceleration sensor 12 and the angular velocity sensor 14. The processing unit 20 which will be described later may perform the temperature correction process instead of the signal processing unit 16, or a function of temperature correction may be incorporated in the acceleration sensor 12 and the angular velocity sensor 14.

The acceleration sensor 12 and the angular velocity sensor 14 may output an analog signal, and in this case, the signal processing unit 16 may perform A/D conversion for an output signal of the acceleration sensor 12 and the angular velocity sensor 14 and generate the sensing data.

The GPS unit 50 (an example of sensor) receives a GPS satellite signal transmitted from a GPS satellite which is a kind of a satellite for positioning, performs positioning calculation using the GPS satellite signal to calculate a position and a speed (vector including magnitude and a direction) of a user in the n frame, and outputs GPS data obtained by adding time information or dilution of precision information to the calculated results to the processing unit 20. A method of calculating a position or a speed or a method of generating time information using the GPS is well known, and therefore, the detailed description thereof will be omitted.

The geomagnetic sensor 60 (an example of sensor) detects geomagnetism in each of three axis directions intersecting each other (ideally, orthogonal to each other) and outputs a digital signal (geomagnetic data) according to the magnitude and the direction of the detected three-axis geomagnetism. However, the geomagnetic sensor 60 may output an analog signal, and in this case, the processing unit 20 may perform A/D conversion for an output signal of the geomagnetic sensor 60 and generate the geomagnetic data.

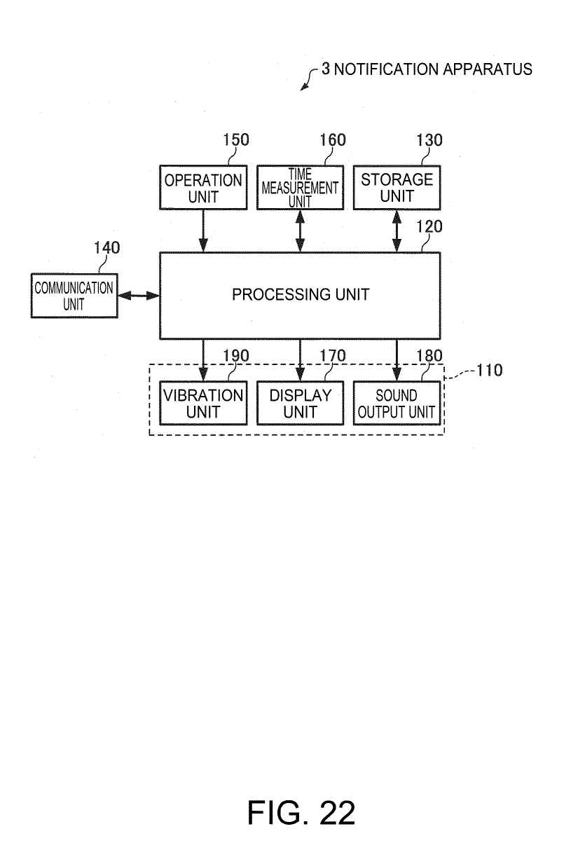

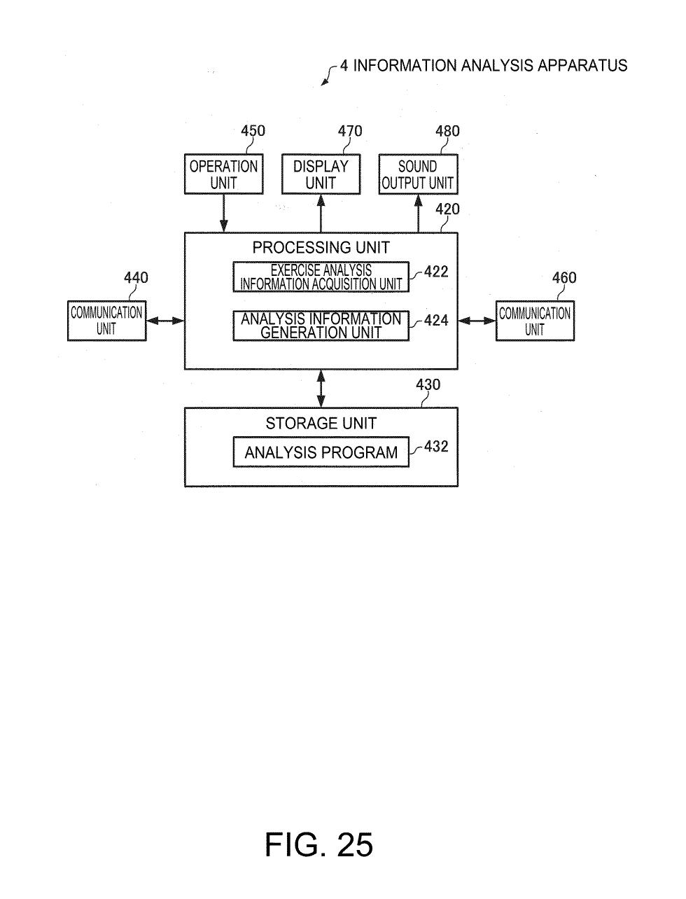

The communication unit 40 is a unit which performs data communication with a communication unit 140 (see FIG. 22) of the notification apparatus 3 or a communication unit 440 (see FIG. 25) of the information analysis apparatus 4, and performs a process of receiving the command (command for starting or finishing the measurement) which is transmitted from the communication unit 140 of the notification apparatus 3 and transmitting the command to the processing unit 20, a process of receiving the running output information or the running result information generated by the processing unit 20 and transmitting the information to the communication unit 140 of the notification apparatus 3, a process of receiving a transmission requesting command for the exercise analysis information from the communication unit 440 of the information analysis apparatus 4 and transmitting the transmission requesting command to the processing unit 20, receiving the exercise analysis information from the processing unit 20, and transmitting the exercise analysis information to the communication unit 440 of the information analysis apparatus 4, and the like.

The processing unit 20 is, for example, configured with a central processing unit (CPU), a digital signal processor (DSP), or an application specific integrated circuit (ASIC), and performs various operation processes or control processes according to various programs stored in the storage unit 30 (recording medium). Particularly, when the command for starting the measurement is received from the notification apparatus 3 through the communication unit 40, the processing unit 20 receives the sensing data, the GPS data, and the geomagnetic data from the inertial measurement unit 10, the GPS unit 50, and the geomagnetic sensor 60 until the command for finishing the measurement is received, and calculates a speed or a position of a user or an attitude angle of the body using these data items. In addition, the processing unit 20 generates various exercise analysis information items which will be described later by performing various operation processes using these calculated information items and analyzing the exercise performed by a user and stores the exercise analysis information in the storage unit 30. Further, the processing unit 20 performs a process of generating the running output information or the running result information using the generated exercise analysis information and transmitting the information items to the communication unit 40.

When the transmission requesting command for the exercise analysis information is received from the information analysis apparatus 4 through the communication unit 40, the processing unit 20 performs a process of reading out the exercise analysis information designated by the transmission requesting command from the storage unit 30 and transmitting the exercise analysis information to the communication unit 440 of the information analysis apparatus 4 through the communication unit 40.

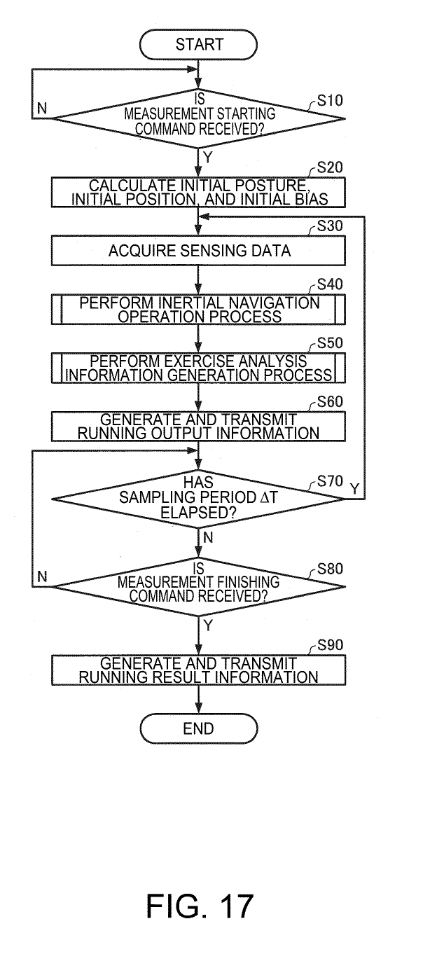

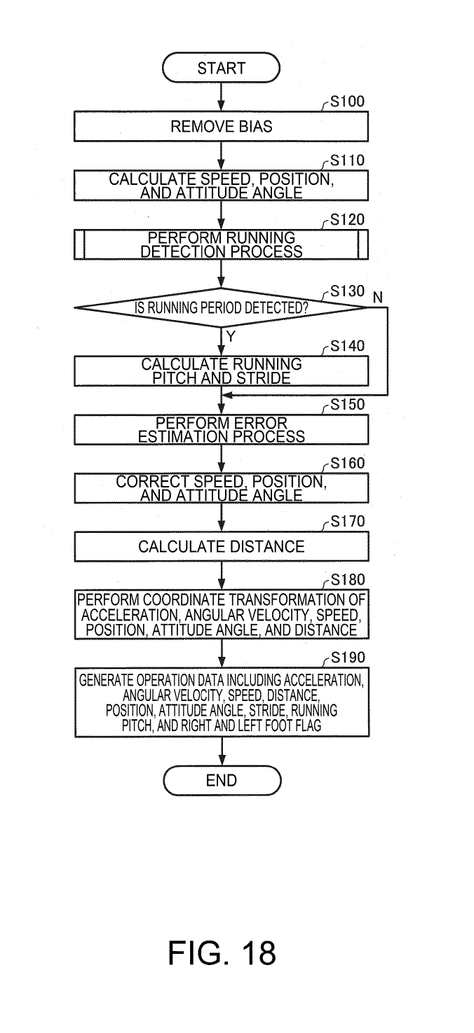

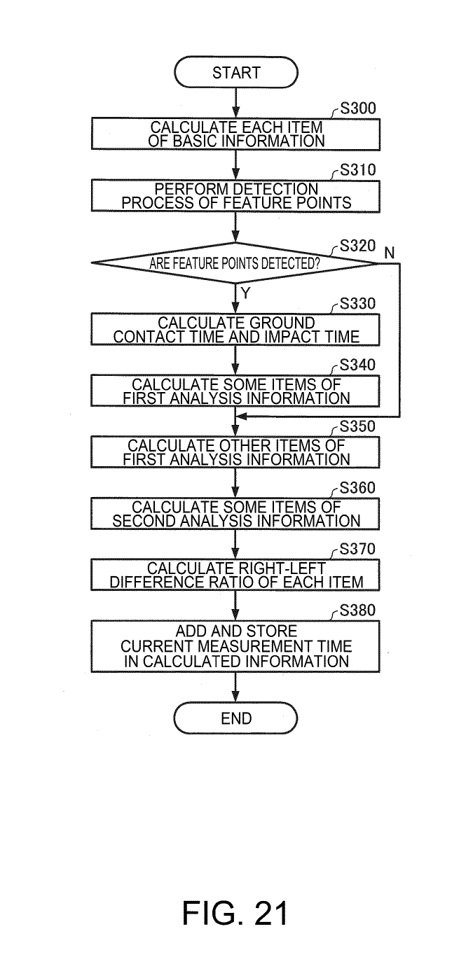

The storage unit 30 is, for example, configured with a recording medium such as a read only memory (ROM), a flash ROM, a hard disk, or a memory card which stores a program or data, or a random access memory (RAM) which is a working area of the processing unit 20. An exercise analysis program 300 which is to be read out by the processing unit 20 for executing the exercise analysis process (see FIG. 17) is stored in the storage unit 30 (any recording medium). The exercise analysis program 300 includes an inertial navigation operation program 302 for executing the inertial navigation operation process (see FIG. 18) and an exercise analysis information generation program 304 for executing an exercise analysis information generation process (see FIG. 21) as subroutines.

In addition, a sensing data table 310, a GPS data table 320, a geomagnetic data table 330, a calculation data table 340, and exercise analysis information 350 are stored in the storage unit 30.

The sensing data table 310 is a data table which stores the sensing data (detection result of the inertial measurement unit 10) received by the processing unit 20 from the inertial measurement unit 10 in time series. FIG. 4 is a diagram showing a configuration example of the sensing data table 310. As shown in FIG. 4, in the sensing data table 310, the sensing data items, in which a detection time 311 of the inertial measurement unit 10, an acceleration 312 detected by the acceleration sensor 12, and an angular velocity 313 detected by the angular velocity sensor 14 are associated with each other, are arranged in time series. When the measurement is started, the processing unit 20 adds a new sensing data item to the sensing data table 310 each time a sampling period Δt (for example, 20 ms or 10 ms) has elapsed. In addition, the processing unit 20 corrects the acceleration and the angular velocity using an acceleration bias and an angular velocity bias estimated by performing an error estimate (which will be described later) using an extended Karman filter, performing overwriting of the corrected acceleration and angular velocity, and updates the sensing data table 310.

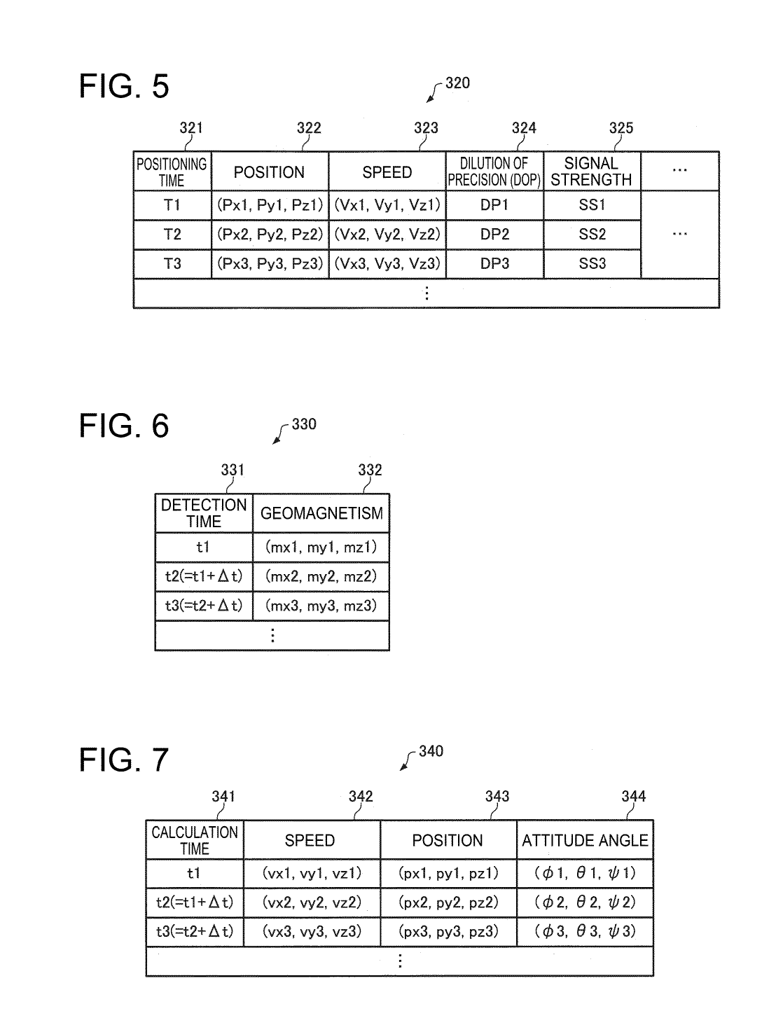

The GPS data table 320 is a data table which stores the GPS data (detected result of the GPS unit 50 (GPS sensor)) received by the processing unit 20 from the GPS unit 50. FIG. 5 is a diagram showing a configuration example of the GPS data table 320. As shown in FIG. 5, in the GPS data table 320, the GPS data items, in which a time 321 obtained by the positioning calculation by the GPS unit 50, a position 322 calculated by performing the positioning calculation, a speed 323 calculated by performing the positioning calculation, and dilution of precision (DOP) 324, and a signal strength 325 of the received GPS satellite signal are associated with each other, are arranged in time series. When the measurement is started, the processing unit 20 adds a new GPS data item each time the GPS data item is acquired (for example, every one second, asynchronously from the acquisition timing of the sensing data item) and updates the GPS data table 320.

The geomagnetic data table 330 is a data table which stores the geomagnetic data (detected result of the geomagnetic sensor 60) received by the processing unit 20 from the geomagnetic sensor 60 in time series. FIG. 6 is a diagram showing a configuration example of the geomagnetic data table 330. As shown in FIG. 6, in the geomagnetic data table 330, the geomagnetic data items, in which a detection time 331 of the geomagnetic sensor 60 and a geomagnetism 332 detected by the geomagnetic sensor 60 are associated with each other, are arranged in time series. When the measurement is started, the processing unit 20 adds a new geomagnetic data item to the geomagnetic data table 330 each time a sampling period Δt (for example, 10 ms) has elapsed.

The calculation data table 340 is a data table which stores the speed, the position, and the attitude angle calculated by the processing unit 20 using the sensing data in time series. FIG. 7 is a diagram showing a configuration example of the calculation data table 340. As shown in FIG. 7, in the calculation data table 340, the calculation data items, in which a time 341, a speed 342, a position 343, and an attitude angle 344 calculated by the processing unit 20 are associated with each other, are arranged in time series. When the measurement is started, the processing unit 20 calculates a speed, a position, and an attitude angle and adds a new calculation data item to the calculation data table 340, each time the sensing data is newly acquired, that is, each time the sampling period Δt has elapsed. In addition, the processing unit 20 corrects the speed, the position, and the attitude angle using a speed error, a position error, and an attitude angle error estimated by performing error estimate using an extended Karman filter, performing overwriting of the corrected speed, position, and attitude angle, and updates the calculation data table 340.

The exercise analysis information 350 includes various information items relating to the exercise performed by a user, and includes each item of input information 351, each item of basic information 352, each item of first analysis information 353, each item of second analysis information 354, and each item of a right-left difference ratio 355 which are generated by the processing unit 20. These various information items will be described later in detail.

### 1-3-2. Functional Configuration of Processing Unit

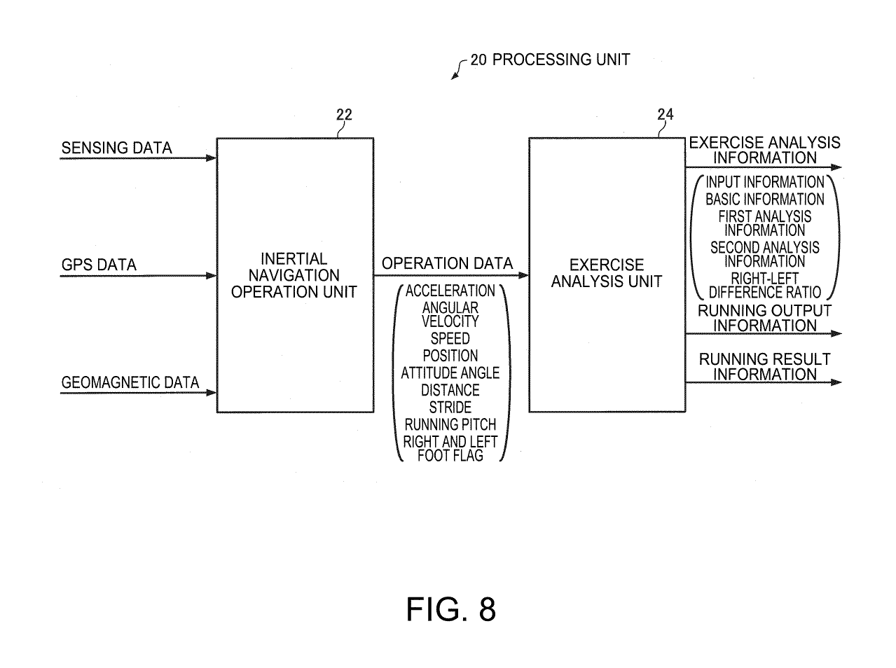

FIG. 8 is a functional block diagram showing a configuration example of the processing unit 20 of the exercise analysis apparatus 2. In the embodiment, the processing unit 20 executes the exercise analysis program 300 stored in the storage unit 30 so as to function as an inertial navigation operation unit 22 and an exercise analysis unit 24. However, the processing unit 20 may receive and execute the exercise analysis program 300 stored in an arbitrary storage device (recording medium) through a network.

The inertial navigation operation unit 22 performs an inertial navigation operation using the sensing data (detected result of the inertial measurement unit 10), the GPS data (detected result of the GPS unit 50), and the geomagnetic data (detected result of the geomagnetic sensor 60), calculates an acceleration, an angular velocity, a speed, a position, an attitude angle, a distance, and a stride and running pitches, and outputs operation data including these calculated results. The operation data to be output by the inertial navigation operation unit 22 is stored in the storage unit 30 in the order of time. The inertial navigation operation unit 22 will be described later in detail.

The exercise analysis unit 24 analyzes an exercise performed by a user at the time of running using the operation data (operation data stored in the storage unit 30) to be output by the inertial navigation operation unit 22, and generates the exercise analysis information (input information, basic information, first analysis information, second analysis information, right-left difference ratio which will be described later) which is information regarding analysis results. The exercise analysis information generated by the exercise analysis unit 24 is stored in the storage unit 30 in the order of time, during the running performed by a user.

In addition, the exercise analysis unit 24 generates the running output information which is information to be output during the running performed by a user (specifically, a period from the start to the end of the measurement performed by the inertial measurement unit 10), using the generated exercise analysis information. The running output information generated by the exercise analysis unit 24 is transmitted to the notification apparatus 3 through the communication unit 40.

The exercise analysis unit 24 generates running result information which is information regarding running results using the exercise analysis information generated during the running, when a user finishes the running (specifically, when the inertial measurement unit 10 finishes the measurement). The running result information generated by the exercise analysis unit 24 is transmitted to the notification apparatus 3 through the communication unit 40.

### 1-3-3. Functional Configuration of Inertial Navigation Operation Unit

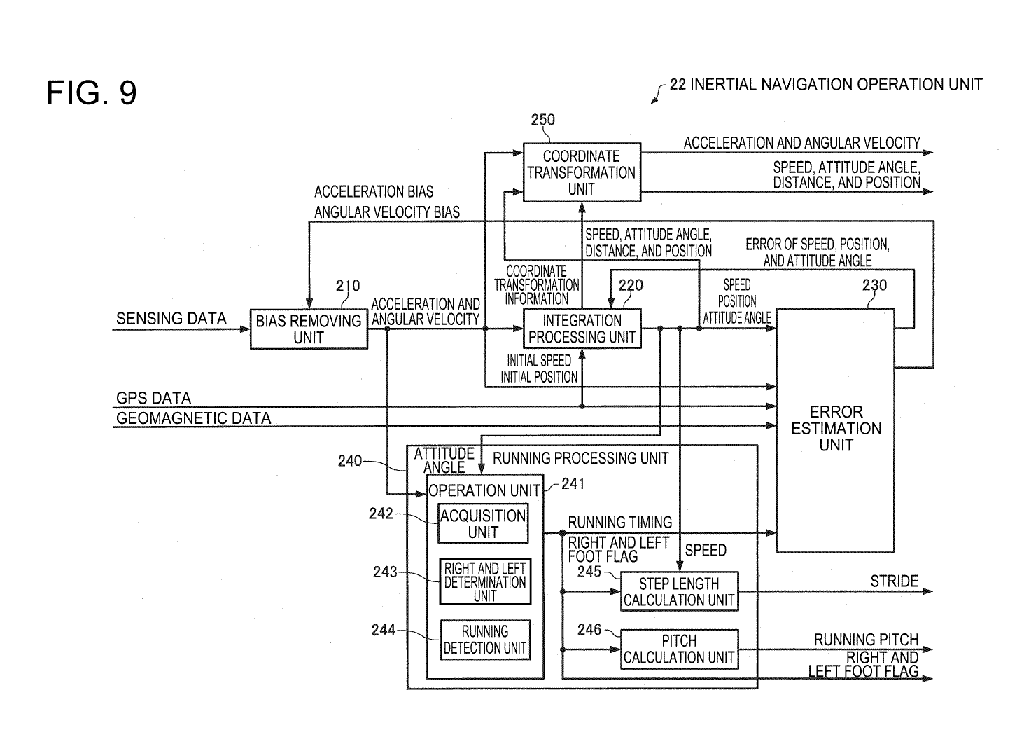

FIG. 9 is a functional block diagram showing a configuration example of the inertial navigation operation unit 22. In the embodiment, the inertial navigation operation unit 22 includes a bias removing unit 210, an integration processing unit 220, an error estimation unit 230, a running processing unit 240, and a coordinate transformation unit 250. However, in the inertial navigation operation unit 22, a part of these constituent elements may be removed or changed or other constituent elements may be added.

The bias removing unit 210 performs a process of subtracting an acceleration bias bₐ and an angular velocity bias b_{ω} estimated by the error estimation unit 230 from each of the three-axis acceleration and the three-axis angular velocity included in the newly acquired sensing data and correcting the three-axis acceleration and the three-axis angular velocity. Since there is no estimated value of the acceleration bias bₐ and the angular velocity bias b_{ω} in an initial state immediately after starting the measurement, the bias removing unit 210 calculates an initial bias using the sensing data from the inertial measurement unit, by assuming an initial state of a user as a resting state.

The integration processing unit 220 performs a process of calculating a speed V^{e}, a position p^{e}, and an attitude angle (a roll angle φ_{be}, a pitch angle θ_{be}, and a yaw angle ψ_{be}) from the acceleration and the angular velocity which are corrected by the bias removing unit 210. Specifically, first, the integration processing unit 220 sets an initial speed as zero by assuming the initial state of a user as the resting state or calculates an initial speed from the speed included in the GPS data, and calculates an initial position from the position included in the GPS data. The integration processing unit 220 calculates initial values of the roll angle φ_{be} and the pitch angle θ_{be} by specifying a gravitational acceleration direction from the three-axis acceleration of the b frame which is corrected by the bias removing unit 210, and calculates an initial value of the yaw angle ψ_{be} from the speed included in the GPS data to set the calculated initial values as initial attitude angles of e frame. When the GPS data is not obtained, the initial value of the yaw angle ψ_{be} is set as zero, for example. The integration processing unit 220 calculates an initial value of a coordinate transformation matrix (rotation matrix) C_{b}^{e} from the b frame to the e frame represented by an equation (1), from the calculated initial attitude angle.

$${C}_{b}^{e} = [ \begin{matrix}cos {θ}_{be} \cdot cos {ϕ}_{be} & cos {θ}_{be} \cdot sin {ϕ}_{be} & - sin {ϕ}_{be} \\ \begin{matrix}sin {φ}_{be} \cdot sin {θ}_{be} \cdot cos {ϕ}_{be} - \\ cos {φ}_{be} \cdot sin {ϕ}_{be}\end{matrix} & \begin{matrix}sin {φ}_{be} \cdot sin {θ}_{be} \cdot sin {ϕ}_{be} + \\ cos {φ}_{be} \cdot cos {ϕ}_{be}\end{matrix} & \begin{matrix}sin {φ}_{be} \cdot \\ cos {θ}_{be}\end{matrix} \\ \begin{matrix}cos {φ}_{be} \cdot sin {θ}_{be} \cdot cos {ϕ}_{be} + \\ sin {φ}_{be} \cdot sin {ϕ}_{be}\end{matrix} & \begin{matrix}cos {φ}_{be} \cdot sin {θ}_{be} \cdot sin {ϕ}_{be} - \\ sin {φ}_{be} \cdot cos {ϕ}_{be}\end{matrix} & \begin{matrix}cos {φ}_{be} \cdot \\ cos {θ}_{be}\end{matrix}\end{matrix} ] \qquad ( 1 )$$

Then, the integration processing unit 220 calculates the coordinate transformation matrix C_{b}^{e} by performing integration (rotation operation) of the three-axis angular velocity which is corrected by the bias removing unit 210 and calculates an attitude angle using an equation (2).

$$[ \begin{matrix}{φ}_{be} \\ {θ}_{be} \\ {ϕ}_{be}\end{matrix} ] = [ \begin{matrix}arctan 2 ( {C}_{ b}^{e} ( 2 , 3 ) , {C}_{b}^{e} ( 3 , 3 ) ) \\ - arcsin {C}_{b}^{e} ( 1 , 3 ) \\ arctan 2 ( {C}_{b}^{e} ( 1 , 2 ) , {C}_{b}^{e} ( 1 , 1 ) )\end{matrix} ] \qquad ( 2 )$$

The integration processing unit 220 converts the three-axis acceleration of the b frame which is corrected by the bias removing unit 210 into a three-axis acceleration of the e frame using the coordinate transformation matrix C_{b}^{e}, and calculates the speed v^{e} of the e frame by performing the integration by removing the gravitational acceleration component. The integration processing unit 220 calculates a position p^{e} of the e frame by performing integration of the speed V^{e} of the e frame.

The integration processing unit 220 performs a process of correcting the speed v^{e}, the position p^{e}, and the attitude angle and a process of calculating a distance by integrating the corrected speed V^{e}, using a speed error δv^{e}, a position error δp^{e}, and an attitude angle error ε^{e} estimated by the error estimation unit 230.

In addition, the integration processing unit 220 also calculates a coordinate transformation matrix C_{b}^{m} from the b frame to the m frame, a coordinate transformation matrix Cₑ^{m} from the e frame to the m frame, and a coordinate transformation matrix Cₑⁿ from the e frame to the n frame. These coordinate transformation matrices are used in the coordinate transformation process of the coordinate transformation unit 250 which will be described later, as coordinate transformation information.

The error estimation unit 230 estimates errors of indexes indicating the state of a user, using the speed or position and attitude angle calculated by the integration processing unit 220, the acceleration or angular velocity corrected by the bias removing unit 210, the GPS data, the geomagnetic data, and the like. In the embodiment, the error estimation unit 230 estimates errors of the speed, the attitude angle, the acceleration, and the angular velocity, and the position using the extended Karman filter. That is, the error estimation unit 230 sets the error (speed error) δv^{e} of the speed V^{e} calculated by the integration processing unit 220, the error (attitude angle error) ε^{e} calculated by the integration processing unit 220, the acceleration bias bₐ and the angular velocity bias b_{ω}, and the error (position error) δp^{e} of the position p^{e} calculated by the integration processing unit 220 as state variables of the extended Karman filter, and a state vector X is defined as shown in an equation (3).

$$X = [ \begin{matrix}δ {v}^{e} \\ {ɛ}^{e} \\ {b}_{a} \\ {b}_{ω} \\ δ {p}^{e}\end{matrix} ] \qquad ( 3 )$$

The error estimation unit 230 predicts the state variables to be included in the state vector X using a prediction expression of the extended Karman filter. The prediction expression of the extended Karman filter is represented as an equation (4). In the equation (4), a matrix Φ is a matrix in which a previous state vector X and a current state vector X are correlated with each other, and some of the elements are designed so as to change every hour while applying the attitude angle or the position. In addition, Q is a matrix representing the process noise and each element thereof is set to an appropriate value in advance. P is an error covariance matrix of the state variables.

X=ΦX

P=ΦPΦ^{T}+Q (4)

The error estimation unit 230 updates (corrects) the predicted state variables using an updating expression of the extended Karman filter. The updating expression of the extended Karman filter is represented as an equation (5). Z and H are respectively an observation vector and an observation matrix, and the updating expression (5) represents that the state vector X is corrected using a difference between the actual observation vector Z and a vector HX predicted from the state vector X. R is a covariance matrix of the observation error, and may be a certain value set in advance or may be dynamically changed. K is Karman gain and K increases as R decreases. In the equation (5), as K increases (R decreases), a corrected amount of the state vector X increases, and P decreases by that amount.

K=PH^{T}(HPH^{T}+R)⁻¹

X=X+K(Z−HX)

P=(I−KH)P (5)

Examples of the error estimation (estimating method of the state vector X) are as follows.

### Error Estimation Method by Performing Correction Based on Attitude Angle Error:

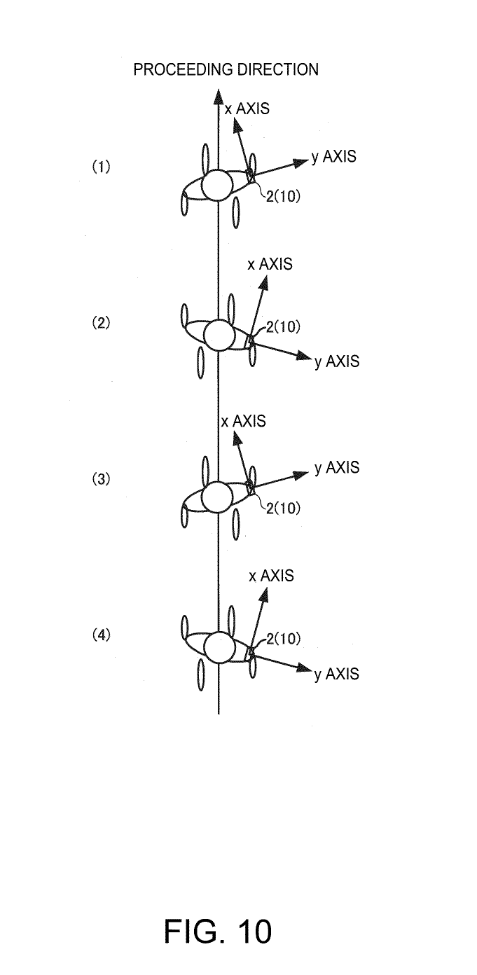

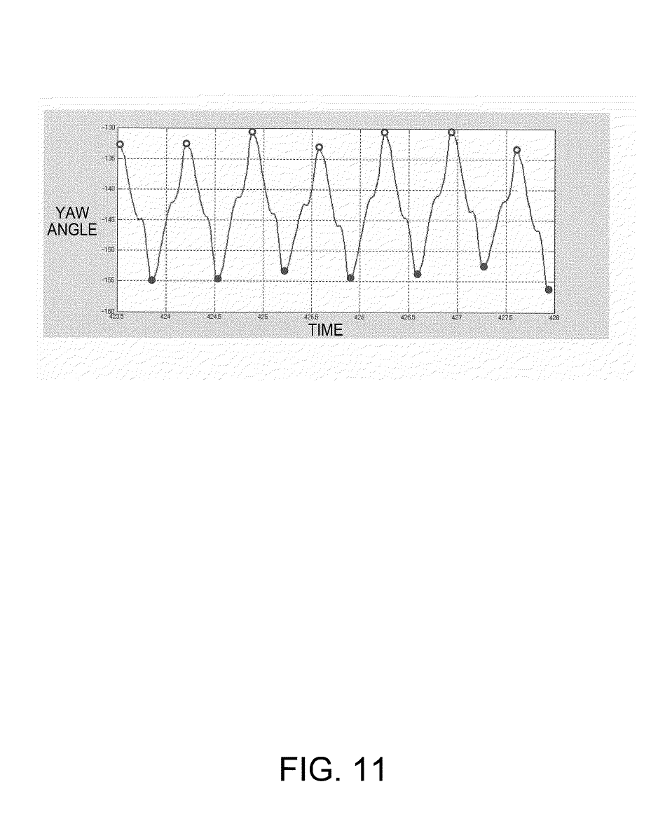

FIG. 10 is a bird's-eye view of movement of a user when a user equipped with the exercise analysis apparatus 2 on the right waist runs (goes straight). FIG. 11 is a diagram showing an example of a yaw angle (azimuth angle) calculated from the detected result of the inertial measurement unit 10 when a user runs (goes straight), in which a horizontal axis indicates time and a vertical axis indicates the yaw angle (azimuth angle).

The position of the inertial measurement unit 10 with respect to a user sometimes changes according to the running operation performed by a user. As shown in (1) or (3) in FIG. 10, in a state where a user took a step with the left foot, the inertial measurement unit 10 takes the position inclined to the left with respect to the proceeding direction (x axis of m frame). With respect to this, as shown in (2) or (4) in FIG. 10, in a state where a user took a step with the right foot, the inertial measurement unit 10 takes the position inclined to the right with respect to the proceeding direction (x axis of m frame). That is, the position of the inertial measurement unit 10 periodically changes for every two steps including one right step and one left step, according to the running operation performed by a user. In FIG. 11, the yaw angle becomes maximum (O in FIG. 11) in a state where a user takes a step with the right foot, and the yaw angle becomes minimum (• in FIG. 11) in a state where a user takes a step with the left foot. Therefore, it is possible to estimate the error by setting the previous (two steps back) attitude angle and the current attitude angle to be equivalent to each other and the previous attitude angle as a true attitude. In this method, the observation vector Z of the equation (5) is a difference between the previous attitude angle and the current attitude angle calculated by the integration processing unit 220, and corrects the state vector X based on a difference between the attitude angle error ε^{e} and the observation value and estimates the error using the updating equation (5).

### Error Estimating Method by Performing Correction Based on Angular Velocity Bias

This is a method of estimating an error by assuming that the previous (two steps back) attitude angle and the current attitude angle are equivalent to each other and the previous attitude angle is not required to be a true attitude. In this method, the observation vector Z of the equation (5) is an angular velocity bias calculated from the previous attitude angle and the current attitude angle calculated by the integration processing unit 220, corrects the state vector X based on a difference between the angular velocity bias b_{ω} and the observation value and estimates the error using the updating equation (5).

### Error Estimating Method by Performing Correction Based on Azimuth Angle Error

This is a method of estimating an error by assuming that the previous (two steps back) yaw angle (azimuth angle) and the current yaw angle (azimuth angle) are equivalent to each other and the previous yaw angle (azimuth angle) is a true yaw angle (azimuth angle). In this method, the observation vector Z is a difference between the previous yaw angle and the current yaw angle calculated by the integration processing unit 220, corrects the state vector X based on a difference between an azimuth angle error ε_{z}^{e} and the observation value and estimates the error using the updating equation (5).

### Error Estimating Method by Performing Correction Based on Stopped State:

This is a method of estimating an error by assuming that a speed is zero in a stopped state. In this method, the observation vector Z is a difference between the speed V^{e} calculated by the integration processing unit 220 and zero, corrects the state vector X based on the speed error δv^{e} and estimates the error using the updating equation (5).

### Error Estimating Method by Performing Correction Based on Resting State:

This is a method of estimating an error by assuming that a speed is zero in the resting state and there is no attitude change. In this method, the observation vector Z is the error of the speed V^{e} calculated by the integration processing unit 220 and a difference between the previous attitude angle and the current attitude angle calculated by the integration processing unit 220, corrects the state vector X based on the speed error δv^{e} and the attitude angle error ε^{e} and estimates the error using the updating equation (5).

### Error Estimating Method by Performing Correction Based on Observation Value of GPS:

This is a method of estimating an error by assuming that the speed V^{e}, the position p^{e}, or the yaw angle ψ_{be} calculated by the integration processing unit 220 and the speed, the position or the azimuth angle (the speed, the position or the azimuth angle after conversion into the e frame) calculated from the GPS data are equivalent to each other. In this method, the observation vector Z is a difference between the speed, the position or the yaw angle calculated by the integration processing unit 220 and the speed, the position or the azimuth angle calculated from the GPS data, corrects the state vector X based on a difference between the speed error δv^{e}, the position error δp^{e}, or the azimuth angle error ε_{z}^{e} and the observation value, and estimates the error using the updating equation (5).

### Error Estimating Method by Performing Correction Based on Observation Value of Geomagnetic Sensor:

This is a method of estimating an error by assuming that the yaw angle ψ_{be} calculated by the integration processing unit 220 and the azimuth angle (azimuth angle after conversion into the e frame) calculated by the geomagnetic sensor 60 are equivalent to each other. In this method, the observation vector Z is a difference between the yaw angle calculated by the integration processing unit 220 and the azimuth angle calculated from the geomagnetic data, corrects the state vector X based on a difference between the azimuth angle error ε_{z}^{e} and the observation value and estimates the error using the updating equation (5).

Returning to FIG. 9, the running processing unit 240 includes an operation unit 241, a step length calculation unit 245, and a pitch calculation unit 246. The operation unit 241 includes an acquisition unit 242, a right and left determination unit 243, and a running detection unit 244.

The acquisition unit 242 acquires detection results from a sensor (for example, inertial measurement unit 10) which is attached to a user and detects information relating to the exercise performed by a user. In the example shown in FIG. 9, the acquisition unit 242 acquires sensing data through the bias removing unit 210.

The right and left determination unit 243 performs a determination process of determining which period the result shows of either a first period including a right foot ground contact period or a second period including a left foot ground contact period, based on the detection result acquired by the acquisition unit 242 (sensing data in the example shown in FIG. 9).

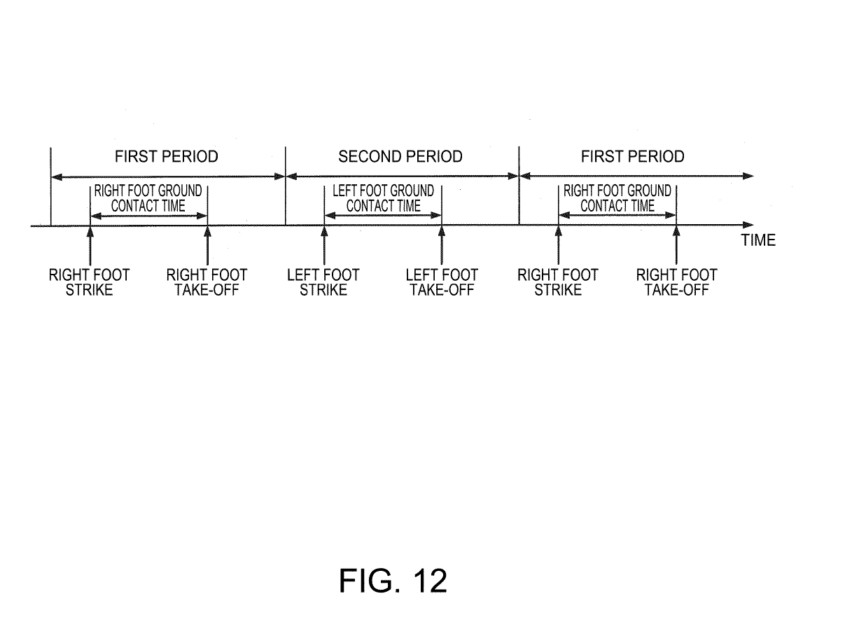

FIG. 12 is a diagram illustrating the first period and the second period. A horizontal axis of FIG. 12 indicates time.

The right foot ground contact period is a period from a timing when the right foot strikes the ground to a timing when the right foot leaves the ground. The left foot ground contact period is a period from a timing when the left foot strikes the ground to a timing when the left foot leaves the ground. The first period is a period including one right foot ground contact period. In addition, the first period is a period not including the left foot ground contact period. The second period is a period including one left foot ground contact period. In addition, the second period is a period not including the right foot ground contact period.

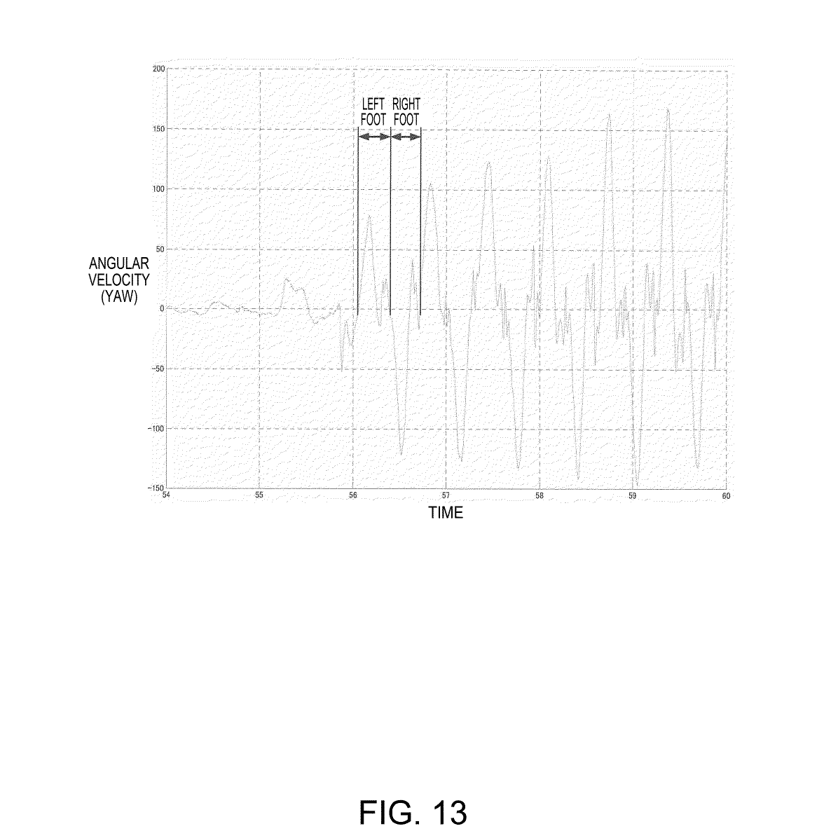

FIG. 13 is a graph showing an angular velocity in a yaw direction. A horizontal axis of FIG. 13 indicates time and a vertical axis thereof indicates an angular velocity in a yaw direction. FIG. 13 shows right rotation when the angular velocity is a positive value, and left rotation when the angular velocity is a negative value.

As shown in FIG. 10, in a top view of a user, the inertial measurement unit 10 rotates clockwise from a state where a user takes a step with the left foot (state of (1) or (3) in FIG. 10) to a state where a user takes a step with the right foot (state (2) or (4) in FIG. 10), and conversely, rotates counterclockwise from a state where a user takes a step with the right foot to a state where a user takes a step with the left foot. Accordingly, the right and left determination unit 243, for example, can determine whether or not the running period is a right or left running period from polarity of the z axis angular velocity (angular velocity in the yaw direction). In addition, the right and left determination unit can also determine whether the running period is a right or left running period, based on the angle of a user in the yaw direction. Since a high-frequency noise component is included in the three-axis angular velocity detected by the inertial measurement unit 10, the right and left determination unit 243 may determine whether or not the running period is a right or left running period using the z axis angular velocity obtained by removing noise through a low-pass filter. Further, the right and left determination unit 243 may perform the determination process, when an absolute value of the z axis angular velocity is equal to or greater than a reference value. The reference value may be approximately 60 degree/sec, for example.

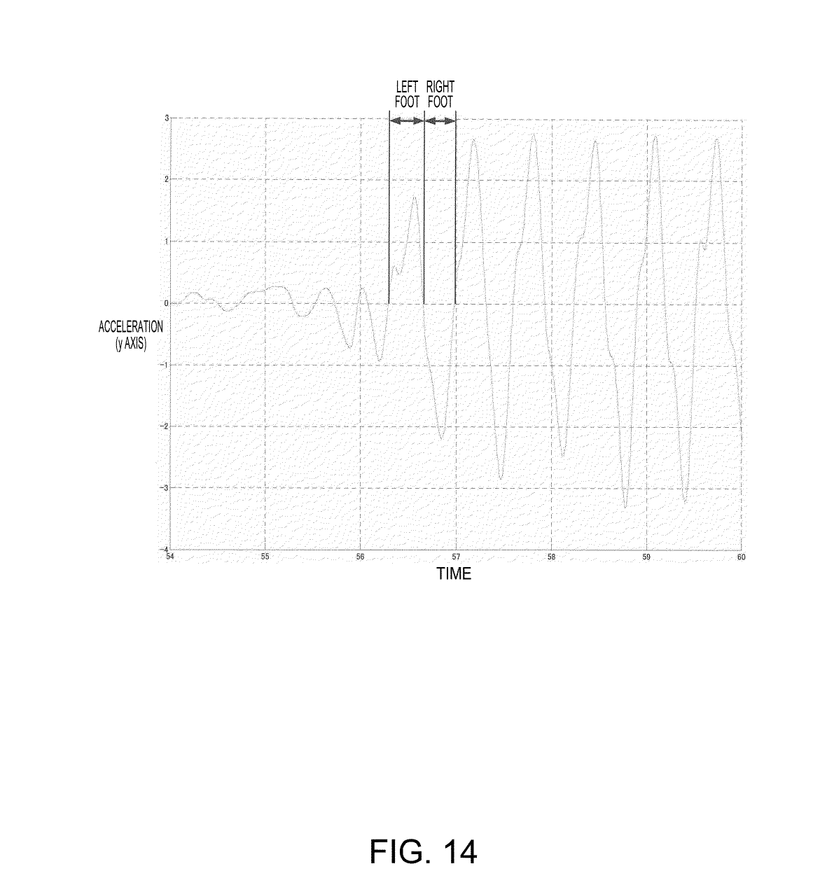

FIG. 14 is a graph showing the y axis acceleration. A horizontal axis of FIG. 14 indicates time and a vertical axis thereof indicates the y axis acceleration. FIG. 14 shows acceleration to the right, when the y axis acceleration is a positive value, and acceleration to the left, when the y axis acceleration is a negative value.

When the right foot comes into contact with the ground, the upper body of a user accelerates to the left. When the left foot comes into contact with the ground, the upper body of a user accelerates to the right. Accordingly, the right and left determination unit 243 can determine whether the running period is a right or left running period from polarity of the y axis acceleration. Since a high-frequency noise component is included in the three-axis acceleration detected by the inertial measurement unit 10, the right and left determination unit 243 may determine whether the running period is a right or left running period using the y axis acceleration obtained by removing noise through a low-pass filter. Further, the right and left determination unit 243 may perform the determination process, when an absolute value of the y axis acceleration is equal to or greater than a reference value.

There is no limitation to these operations, and the right and left determination unit 243 may perform the determination process based on the y axis speed or attitude angle (yaw direction), for example.

The right and left determination unit 243 outputs a right and left foot flag (for example, on in a case of the right foot and off in a case of the left foot) indicating the right or left running period, according to the results of the determination process.

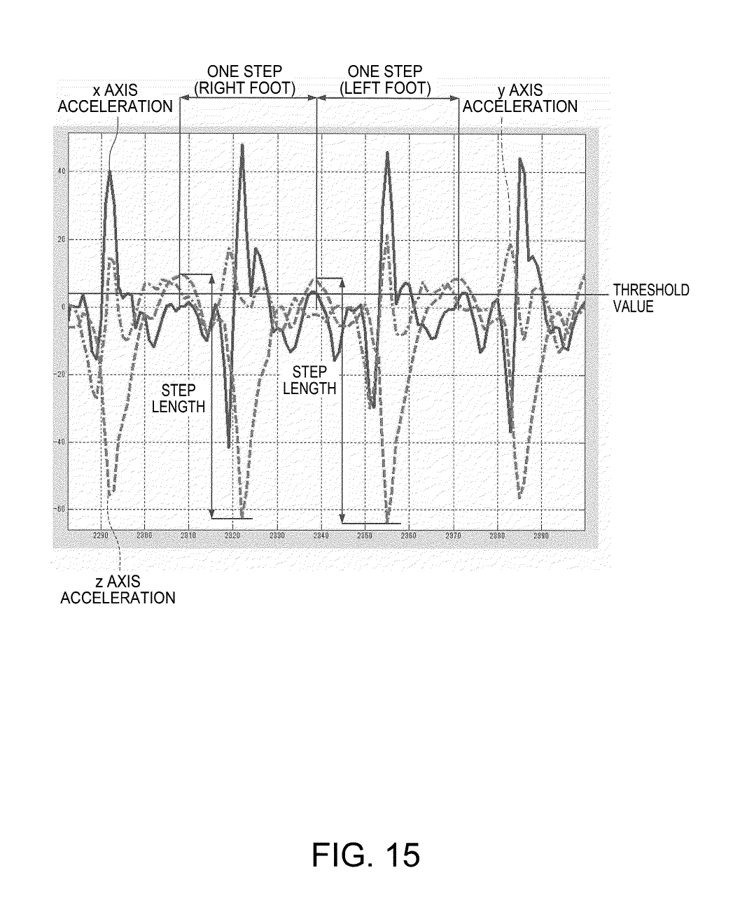

Returning to FIG. 9, the running detection unit 244 performs a process of detecting a running period (running timing) of a user by using the detected result of the inertial measurement unit 10 (specifically, sensing data corrected by the bias removing unit 210). As described in FIG. 10 and FIG. 11, the posture of a user changes periodically (for every two steps (one right step and one left step)) at the time when running is performed by a user, and accordingly, an acceleration to be detected by the inertial measurement unit 10 also changes periodically. FIG. 15 is a diagram showing an example of the three-axis acceleration detected by the inertial measurement unit 10 at the time when running is performed by a user. In FIG. 15, a horizontal axis indicates the time and a vertical axis indicates the acceleration. As shown in FIG. 15, the three-axis acceleration periodically changes, and it is found that the z axis (axis of gravity direction) acceleration particularly changes regularly with periodicity. This z axis acceleration reflects an acceleration in the vertical motion of a user, and a period from the time when the z axis acceleration becomes a maximum value equal to or greater than a predetermined threshold value to the time when the z axis acceleration becomes a maximum value equal to or greater than a predetermined threshold value the next time corresponds to a period of one step.

Therefore, in the embodiment, the running detection unit 244 detects a running period, each time the z axis acceleration (which corresponds to an acceleration in the vertical motion of a user) detected by the inertial measurement unit 10 becomes a maximum value equal to or greater than a predetermined threshold value. That is, the running detection unit 244 outputs a timing signal indicating the detection of the running period, each time the z axis acceleration becomes a maximum value equal to or greater than a predetermined threshold value. In practice, since a high-frequency noise component is included in the three-axis acceleration detected by the inertial measurement unit 10, the running detection unit 244 detects the running period using the z axis acceleration obtained by removing noise through a low-pass filter.

The right and left determination unit 243 may perform the determination process, based on the detection results of the inertial measurement unit 10 and the running timing detected by the running detection unit 244. Accordingly, it is possible to precisely grasp the exercise motion of the right and left foot.

The step length calculation unit 245 performs a process of calculating a step length for each right and left step using the timing signal of the running period and the right and left foot flag output by the running detection unit 244 and the speed or the position calculated by the integration processing unit 220, and outputting the calculated step lengths as strides for each of the right and left steps. That is, the step length calculation unit 245 calculates the step length by integrating the speed for each sampling period Δt in the period from the start of the running period to the start of the next running period (by calculating a difference between the position at the time of start of the running period and the position at the time of next start of the running period), and outputs the step length as a stride.

The pitch calculation unit 246 performs a process of calculating the number of steps in one minute using the timing signal of the running period output by the running detection unit 244 and outputting the calculated number of steps as a running pitch. That is, the pitch calculation unit 246, for example, calculates the number of steps per second by obtaining an inverse number of the running period, and calculates the number of steps in one minute (running pitch) by multiplying 60 by the calculated number of steps per second.

The coordinate transformation unit 250 performs a coordinate transformation process of respectively transforming the three-axis acceleration and the three-axis angular velocity of the b frame which are corrected by the bias removing unit 210 into the three-axis acceleration and the three-axis angular velocity of the m frame, using the coordinate transformation information (coordinate transformation matrix C_{b}^{m}) from the b frame to the m frame which is calculated by the integration processing unit 220. The coordinate transformation unit 250 performs a coordinate transformation process of respectively transforming the speed in the three-axis direction, the attitude angle around three axes, the distance in the three-axis direction of the e frame which are calculated by the integration processing unit 220 into the speed in the three-axis direction, the attitude angle around three axes, and the distance in the three-axis direction of the m frame, using the coordinate transformation information (coordinate transformation matrix Cₑ^{m}) from the e frame to the m frame which is calculated by the integration processing unit 220. The coordinate transformation unit 250 performs a coordinate transformation process of transforming the position of the e frame which is calculated by the integration processing unit 220 into the position of the n frame, using the coordinate transformation information (coordinate transformation matrix Cₑⁿ) from the e frame to the n frame which is calculated by the integration processing unit 220.

The inertial navigation operation unit 22 outputs (stores) operation data including information items such as the acceleration, the angular velocity, the speed, the position, the attitude angle, and the distance after the coordinate transformation performed by the coordinate transformation unit 250 and the stride, the running pitch, and the right and left foot flag calculated by the running processing unit 240 (in the storage unit 30).

### 1-3-4. Functional Configuration of Exercise Analysis Unit

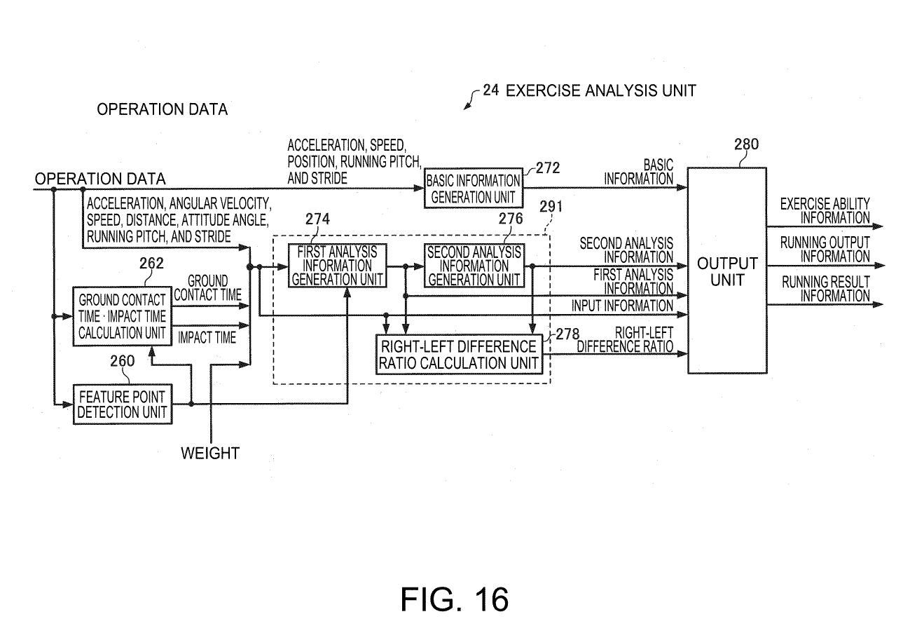

FIG. 16 is a functional block diagram showing a configuration example of the exercise analysis unit 24. In the embodiment, the exercise analysis unit 24 includes a feature point detection unit 260, a ground contact time•impact time calculation unit 262, a basic information generation unit 272, a calculation unit 291, and an output unit 280. However, in the exercise analysis unit 24, a part of these constituent elements may be removed or changed or other constituent elements may be added.

The feature point detection unit 260 performs a process of detecting feature points of the running exercise performed by a user using the operation data. The feature points of the running exercise performed by a user are, for example, strike (the time when a part of the sole touches the ground, the time when the entire sole touches the ground, an arbitrary time while the heel touches the ground and the tiptoe is lifted, an arbitrary time while the tiptoe touches the ground and the heel is lifted, or the period when the entire sole touches the ground may be suitably set), mid-stance (a state where the weight is loaded onto the foot at maximum), lifting (also referred to as take-off, the time when a part of the sole is lifted from the ground, the time when the entire sole is lifted from the ground, an arbitrary time while the heel touches the ground and the tiptoe is lifted, or an arbitrary time while the tiptoe touches the ground and the heel is lifted may be suitably set), and the like. Specifically, the feature point detection unit 260 separately detects feature points in the running period of the right foot and the feature points in the running period of the left foot by using the right and left foot flag included in the operation data. The feature point detection unit 260 can detect the strike when the acceleration in the vertical direction (detected value of z axis of the acceleration sensor 12) is changed from a positive value to a negative value, detect the mid-stance when the acceleration in the proceeding direction becomes a peak after the acceleration in the vertical direction becomes a peak in a negative direction after the strike, and detect the lifting (take-off) when the acceleration in the vertical direction is changed from a negative value to a positive value.

The ground contact time•impact time calculation unit 262 performs a process of calculating each value of the ground contact time and the impact time with the time when the feature point detection unit 260 has detected the feature points as a reference using the operation data. Specifically, the ground contact time•impact time calculation unit 262 determines whether the current operation data is the running period of the right foot or the running period of the left foot from the right and left foot flag included in the operation data, and calculates each value of the ground contact time and the impact time for the running period of the right foot or the running period of the left foot, using the time when the feature point detection unit 260 has detected the feature points as a reference. The definitions and calculating method of the ground contact time and the impact time will be described later in detail.

The basic information generation unit 272 performs a process of generating basic information relating to the exercise of a user, using information items including the acceleration, the speed, the position, the stride, and the running pitch included in the operation data. Herein, the basic information includes each item of a running pitch, a stride, a running speed, an altitude, a running distance, and a running time (lap time). Specifically, the basic information generation unit 272 outputs the running pitch and the stride included in the operation data as the running pitch and the stride of the basic information. The basic information generation unit 272 calculates current values of a running speed, an altitude, a running distance, and a running time (lap time) and an average value thereof at the time of running, by using some or all of the acceleration, the speed, the position, the running pitch, and the stride included in the operation data.

The calculation unit 291 calculates indexes relating to the exercise performed by a user. The calculation unit 291 may calculate an index in the first period and an index in the second period. For example, the calculation unit 291 may calculate a difference between the index in the first period and the index in the second period. In the embodiment, the calculation unit 291 includes a first analysis information generation unit 274, a second analysis information generation unit 276, and a right-left difference ratio calculation unit 278.

The first analysis information generation unit 274 performs a process of analyzing the exercise performed by a user with the timing when the feature point detection unit 260 has detected the feature points as a reference, using the input information and generating first analysis information.

Herein, the input information includes each item of an acceleration in the proceeding direction, a speed in the proceeding direction, a distance in the proceeding direction, an acceleration in the vertical direction, a speed in the vertical direction, a distance in the vertical direction, an acceleration in the horizontal direction, a speed in the horizontal direction, a distance in the horizontal direction, an attitude angle (roll angle, pitch angle, and yaw angle), an angular velocity (roll direction, pitch direction, yaw direction), a running pitch, a stride, a ground contact time, an impact time, and weight. The weight is input by a user, the contact time and the impact time are calculated by the ground contact time•impact time calculation unit 262, and the other items are included in the operation data.

The first analysis information includes each item of a strike time deceleration amount (a strike time deceleration amount 1 and a strike time deceleration amount 2), a direct below strike ratio (a direct below strike ratio 1, a direct below strike ratio 2, and a direct below strike ratio 3), propulsion (propulsion 1 and propulsion 2), propulsive efficiency (propulsive efficiency 1, propulsive efficiency 2, propulsive efficiency 3, and propulsive efficiency 4), exercise energy, a strike impact, a running ability, a forward inclination angle, a degree of timing coincidence, and leg turnover. Each item of the first analysis information is an item indicating the running state (an example of exercise state) of a user. The definition and a calculating method of each item of the first analysis information will be described later in detail.

The first analysis information generation unit 274 calculates a value of each item of the first analysis information for the right and left sides of the body of a user. Specifically, the first analysis information generation unit 274 calculates each item included in the first analysis information for the running period of the right foot or the running period of the left foot, according to whether the feature point detection unit 260 has detected the feature points in the running period of the right foot or the feature points in the running period of the left foot. The first analysis information generation unit 274 also calculates an average value of right and left sides and the total value regarding each item included in the first analysis information.

A second analysis information generation unit 276 performs a process of generating second analysis information using the first analysis information generated by the first analysis information generation unit 274. Herein, the second analysis information includes each item of energy loss, energy efficiency, and a burden on the body. The definition and a calculating method of each item of the second analysis information will be described later in detail. The second analysis information generation unit 276 calculates a value of each item of the second analysis information for the running period of the right foot or the running period of the left foot. The second analysis information generation unit 276 also calculates an average value of right and left sides and the total value regarding each item included in the second analysis information.

The right-left difference ratio calculation unit 278 performs a process of calculating a right-left difference ratio which is an index indicating a right and left balance of the body of a user using the value of the running period of the right foot or the running period of the left foot, regarding the running pitch, the stride, the ground contact time, and the impact time included in the input information, all items of the first analysis information and all items of the second analysis information. The definition and a calculating method of each item of the right-left difference ratio will be described later.

When a difference between the index in the first period and the index in the second period is equal to or greater than a reference value, the output unit 280 outputs difference information which is information relating to the difference therebetween. The output unit 280, for example, may output information relating to the index having a difference equal to or greater than a reference value, as the difference information.

In addition, the output unit 280 performs a process of generating and outputting the running output information which is information output during the running performed by a user, using the basic information, the input information, the first analysis information, the second analysis information, and the right-left difference ratio. The “running pitch”, the “stride”, the “ground contact time”, and the “impact time” included in the input information, all items of the first analysis information, all items of the second analysis information, and the right-left difference ratio are exercise indexes used for evaluation of a running technique of a user, and the running output information includes information regarding some or all values of these exercise indexes. The exercise indexes included in the running output information may be determined in advance or may be selected by a user by operating the notification apparatus 3. The running output information may include some or all of the running speed, the altitude, the running distance, and the running time (lap time) included in the basic information.

The output unit 280 generates the running result information which is information regarding the running result of a user, using the basic information, the input information, the first analysis information, the second analysis information, and the right-left difference ratio. The output unit 280, for example, may generate the running result information including information regarding an average value of each exercise index during the running performed by a user (the measurement performed by the inertial measurement unit 10). The running result information may include some or all of the running speed, the altitude, the running distance, and the running time (lap time). The output unit 280 transmits the running output information to the notification apparatus 3 through the communication unit 40 during the running performed by a user, and transmits the running result information to the notification apparatus 3 when a user finishes running.

### 1-3-5. Input Information

Hereinafter, each item of the input information will be described in detail. Acceleration in Proceeding Direction, Acceleration in Vertical Direction, and Acceleration in Horizontal Direction

The “proceeding direction” is a proceeding direction of a user (x axis direction of the m frame), the “vertical direction” is a perpendicular direction (z axis direction of the m frame), and the “horizontal direction” is a direction orthogonal to the proceeding direction and the vertical direction (y axis direction of the m frame). The acceleration in the proceeding direction, the acceleration in the vertical direction, and the acceleration in the horizontal direction are respectively an acceleration in the x axis direction, an acceleration in the z axis direction, and an acceleration in the y axis direction of the m frame, and are calculated by the coordinate transformation unit 250.

### Speed in Proceeding Direction, Speed in Vertical Direction, and Speed in Horizontal Direction

The speed in the proceeding direction, the speed in the vertical direction, and the speed in the horizontal direction are respectively a speed in the x axis direction, a speed in the z axis direction, and a speed in the y axis direction of the m frame, and are calculated by the coordinate transformation unit 250. Alternatively, the speed in the proceeding direction, the speed in the vertical direction, and the speed in the horizontal direction can also be calculated by integrating the respective acceleration in the proceeding direction, the acceleration in the vertical direction, and the acceleration in the horizontal direction.

### Angular Velocity (Roll Direction, Pitch Direction, and Yaw Direction)

The angular velocity in the roll direction, the angular velocity in the pitch direction, and the angular velocity in the yaw direction are respectively an angular velocity around the x axis, an angular velocity around the y axis, and an angular velocity around the z axis of the m frame, and are calculated by the coordinate transformation unit 250.

### Attitude Angle (Roll Angle, Pitch Angle, and Yaw Angle)

The roll angle, the pitch angle, and the yaw angle are respectively an attitude angle around the x axis, an attitude angle around the y axis, and an attitude angle around the z axis of the m frame output from the coordinate transformation unit 250, and are calculated by the coordinate transformation unit 250. Alternatively, the roll angle, the pitch angle, and the yaw angle can also be calculated by integrating (performing rotation operation) of the angular velocity in the roll direction, the angular velocity in the pitch direction, and the angular velocity in the yaw direction.

### Distance in Proceeding Direction, Distance in Vertical Direction, and Distance in Horizontal Direction

The distance in the proceeding direction, the distance in the vertical direction, and the distance in the horizontal direction are a traveling distance in the x axis direction, a traveling distance in the z axis direction, and a traveling distance in the y axis direction of the m frame, from a desired position (for example, a position immediately before the user starts running), and are calculated by the coordinate transformation unit 250.

### Running Pitch

The running pitch is an exercise index defined as the number of steps per minute and is calculated by the pitch calculation unit 246. Alternatively, the running pitch can also be calculated by dividing the distance in the proceeding direction in one minute by the stride.

### Stride

The stride is an exercise index defined as the step length of one step and is calculated by the step length calculation unit 245. Alternatively, the stride can also be calculated by dividing the distance in the proceeding direction for one minute by the running pitch.

### Ground Contact Time

The ground contact time is an exercise index defined as the time taken from the strike to the lifting (take-off) and is calculated by the ground contact time•impact time calculation unit 262. The lifting (take-off) means the time when the tiptoe is lifted from the ground. Since the ground contact time has high correlation with the running speed, the ground contact time can also be used as the running ability of the first analysis information.

### Impact Time

The impact time is an exercise index defined as the time when an impact which is generated by the strike is applied to the body, and is calculated by the ground contact time•impact time calculation unit 262. The impact time can be calculated with an expression calculated as (time when the acceleration in the proceeding direction during one step becomes minimum−time of strike).

### Weight

The weight is the weight of a user and a numerical value thereof is input by a user operating an operation unit 150 (see FIG. 18) before running.

### 1-3-6. First Analysis Information

Hereinafter, each item of the first analysis information calculated by the first analysis information generation unit 274 will be described in detail.

### Strike Time Deceleration Amount 1

The strike time deceleration amount 1 is an exercise index defined as an amount of the speed decreased due to the strike, and the strike time deceleration amount 1 can be calculated with an expression of (speed in the proceeding direction before strike−lowest speed in the proceeding direction after strike). The speed in the proceeding direction decreases due to the strike, and the lowest point of the speed in the proceeding direction during one step after the strike is the lowest speed in the proceeding direction.

### Strike Time Deceleration Amount 2

The strike time deceleration amount 2 is an exercise index defined as the lowest negative acceleration in the proceeding direction which is generated due to the strike and coincides with the lowest acceleration in the proceeding direction of one step after the strike. The lowest point of the acceleration in the proceeding direction during one step after the strike is the lowest acceleration in the proceeding direction.

### Direct Below Strike Ratio 1

The direct below strike ratio 1 is an exercise index indicating whether the foot strikes directly below the body. When the foot strikes directly below the body, the deceleration amount at the time of strike decreases, and a user can run efficiently. Since the normal deceleration amount increases according to the speed, only the deceleration amount is not sufficient as an index, but the direct below strike ratio 1 is an index represented as a ratio, and therefore, according to the direct below strike ratio 1, the same evaluation can be performed, even when the speed is changed. When a relationship of α=arctan (acceleration in the proceeding direction at the time of strike/acceleration in the vertical direction at the time of strike) using the acceleration in the proceeding direction (negative acceleration) and the acceleration in the vertical direction at the time of strike is satisfied, the direct below strike ratio 1 can be calculated with an expression of cos α×100(%). Alternatively, an ideal angle α′ is calculated using data items of a plurality of fast runners, and the direct below strike ratio 1 can also be calculated with an expression of {1−|(α′−α)/α′|}×100(%).

### Direct Below Strike Ratio 2

The direct below strike ratio 2 is an exercise index indicating whether the foot strikes directly below the body, using a degree of a decrease of the speed at the time of strike, and the direct below strike ratio 2 can be calculated with an expression of (lowest speed in the proceeding direction after strike/speed in the proceeding direction immediately before strike)×100(%).

### Direct Below Strike Ratio 3

The direct below strike ratio 3 is an exercise index indicating whether the foot strikes directly below the body, using a distance or time from the strike to a state where the foot reaches directly below the body. The direct below strike ratio 3 can be calculated with an expression of (distance in the proceeding direction when the foot reaches directly below the body−distance in the proceeding direction at the time of strike) or can be calculated with an expression of (time when the foot reaches directly below the body−the time of strike). There is a timing when the acceleration in the vertical direction becomes a peak in the negative direction after the strike (point where the acceleration in the vertical direction changes from a positive value to a negative value), and this timing can be determined as a timing (time) when the foot reaches directly below the body.

In addition, the direct below strike ratio 3 may be defined with an equation of the direct below strike ratio 3=arctan (distance from the strike to a state where the foot reaches directly below the body/height of the waist). Alternatively, the direct below strike ratio 3 may be defined with an equation of the direct below strike ratio 3=(1−distance from the strike to a state where the foot reaches directly below the body/traveling distance from the strike to take-off)×100(%) (a ratio of a distance from the strike to a state where the foot reaches directly below the body occupied in the traveling distance while the foot comes in contact with the ground). Alternatively, the direct below strike ratio 3 may be defined with an equation of the direct below strike ratio 3=(1−time from the strike to a state where the foot reaches directly below the body/travel time from the strike to take-off)×100(%) (a ratio of the time from the strike to a state where the foot reaches directly below the body occupied in the travel time while the foot comes in contact with the ground).

### Propulsion 1

The propulsion 1 is an exercise index defined as an amount of the speed which is increased in the proceeding direction due to the take-off from the ground by a user, and the propulsion 1 can be calculated with an expression of (highest speed in the proceeding direction after take-off−lowest speed in the proceeding direction before take-off).

### Propulsion 2

The propulsion 2 is an exercise index defined as a maximum acceleration in the positive proceeding direction generated due to the take-off and coincides with the maximum acceleration in the proceeding direction of one step after the take-off.

### Propulsive Efficiency 1

The propulsive efficiency 1 is an exercise index indicating whether a take-off force efficiently becomes the propulsion. If there is no unnecessary vertical motion and no unnecessary horizontal motion, a user can run efficiently. Since the normal vertical motion and horizontal motion increase according to the speed, only the normal vertical motion and horizontal motion are not sufficient as an index, however, the propulsive efficiency 1 is an exercise index represented as a ratio, and therefore, according to the propulsive efficiency 1, the same evaluation can be performed, even when the speed is changed. The propulsive efficiency 1 is calculated in the vertical direction and the horizontal direction, respectively. When an expression of γ=arctan (acceleration in the vertical direction at the time of take-off/acceleration in the proceeding direction at the time of take-off) is set using the acceleration in the vertical direction and the acceleration in the proceeding direction at the time of take-off, the propulsive efficiency 1 in the vertical direction can be calculated with an expression of cos γ×100(%). Alternatively, an ideal angle γ′ is calculated using data items of a plurality of fast runners and the propulsive efficiency 1 in the vertical direction can be calculated with an expression of {1−(γ′−γ)/γ′|}×100(%). In the same manner as described above, when an expression of δ=arctan (acceleration in the horizontal direction at the time of take-off/acceleration in the proceeding direction at the time of take-off) is set using the acceleration in the horizontal direction and the acceleration in the proceeding direction at the time of take-off, the propulsive efficiency 1 in the horizontal direction can be calculated with an expression of cos δ×100(%). Alternatively, an ideal angle δ′ is calculated using data items of a plurality of fast runners and the propulsive efficiency 1 in the horizontal direction can be calculated with an expression of {1−|(δ′−δ)/δ′|}×100(%).

In addition, the propulsive efficiency 1 in the vertical direction can also be calculated by substituting γ with arctan (speed in the vertical direction at the time of take-off/speed in the proceeding direction at the time of take-off). In the same manner as described above, the propulsive efficiency 1 in the horizontal direction can also be calculated by substituting δ with arctan (speed in the horizontal direction at the time of take-off/speed in the proceeding direction at the time of take-off).

### Propulsive Efficiency 2

The propulsive efficiency 2 is an exercise index indicating whether a take-off force efficiently becomes the propulsion using an angle of the acceleration in mid-stance. Regarding the propulsive efficiency 2 in the vertical direction, when an expression of ξ=arctan (acceleration in the vertical direction in mid-stance/acceleration in the proceeding direction in mid-stance) is set using the acceleration in the vertical direction and the acceleration in the proceeding direction in mid-stance, the propulsive efficiency 2 in the vertical direction can be calculated with an expression of cos ξ×100(%). Alternatively, an ideal angle ξ′ is calculated using data items of a plurality of fast runners and the propulsive efficiency 2 in the vertical direction can be calculated with an expression of {1−|(ξ′−ξ)/ξ′|}×100(%). In the same manner as described above, when an expression of η=arctan (acceleration in the horizontal direction in mid-stance/acceleration in the proceeding direction in mid-stance) is set using the acceleration in the horizontal direction and the acceleration in the proceeding direction in mid-stance, the propulsive efficiency 2 in the horizontal direction can be calculated with an expression of cos η×100(%). Alternatively, an ideal angle η′ is calculated using data items of a plurality of fast runners and the propulsive efficiency 2 in the horizontal direction can be calculated with an expression of {1−|(η′−η)/η′|}×100(%).

In addition, the propulsive efficiency 2 in the vertical direction can also be calculated by substituting ξ with arctan (speed in the vertical direction in mid-stance/speed in the proceeding direction in mid-stance). In the same manner as described above, the propulsive efficiency 2 in the horizontal direction can also be calculated by substituting η with arctan (speed in the horizontal direction in mid-stance/speed in the proceeding direction in mid-stance).

### Propulsive Efficiency 3

The propulsive efficiency 3 is an exercise index indicating whether a take-off force efficiently becomes the propulsion, using a running angle. When the highest point of one step in the vertical direction (½ of the step length of the distance in the vertical direction) is set as H and the distance in the proceeding direction from the take-off to the strike is set as X, the propulsive efficiency 3 can be calculated using an equation (6).

$$Propulsive Efficiency 3 = arcsin ( \sqrt{\frac{16 {H}^{2}}{{X}^{2} + 16 {H}^{2}}} ) \qquad ( 6 )$$

### Propulsive Efficiency 4

The propulsive efficiency 4 is an exercise index indicating whether a take-off force efficiently becomes the propulsion, using a ratio of energy used for proceeding in the proceeding direction relative to the total energy generated during one step, and the propulsive efficiency 4 can be calculated with an expression of (energy used for proceeding in the proceeding direction/energy used for one step)×100(%). This energy is the sum of positional energy and exercise energy.

### Exercise Energy

The exercise energy is an exercise index which is defined as an amount of energy consumed for proceeding one step and also indicates a value obtained by integrating the amount of energy consumed for proceeding one step in the running period. The exercise energy can be calculated with an expression of the exercise energy=(amount of energy consumption in the vertical direction+amount of energy consumption in the proceeding direction+amount of energy consumption in the vertical direction). Herein, the amount of energy consumption in the vertical direction can be calculated with an expression of (weight×gravity×distance in the vertical direction). The amount of energy consumption in the proceeding direction can be calculated with an expression of [weight×{(highest speed in the proceeding direction after take-off)₂−(lowest speed in the proceeding direction after strike)²}/2]. The amount of energy consumption in the horizontal direction can be calculated with an expression of [weight×{(highest speed in the horizontal direction after take-off)²−(lowest speed in the horizontal direction after strike)²}/2].

### Strike Impact

The strike impact is an exercise index indicating a degree of an impact applied to the body due to the strike and the strike impact is calculated with an expression of (impact force in the vertical direction+impact force in the proceeding direction+impact force in the horizontal direction). Herein, the impact force in the vertical direction is calculated with an expression of (weight x the speed in the vertical direction at the time of strike/impact time). The impact force in the proceeding direction is calculated with an expression of {weight x (the speed in the proceeding direction before strike−lowest speed in the proceeding direction after strike)/impact time}. The impact force in the horizontal direction is calculated with an expression of {weight×(speed in the horizontal direction before strike−lowest speed in the horizontal direction after strike)/impact time}.

### Running Ability

The running ability is an exercise index indicating the ability that a user has to run. For example, a correlation between a ratio of the stride and the ground contact time and a record running (time) is known (“Ground contact time and lifting time in a 100 m race” in Journal of Research and Development for Future Athletics 3(1):1-4, 2004), and the running ability is calculated with an expression of (stride/ground contact time).

### Forward Inclination Angle

The forward inclination angle is an exercise index indicating a degree to which the body of a user is inclined with respect to the ground. A forward inclination angle in a state where a user stands vertically with respect to the ground is set as 0 degrees, a forward inclination angle when a user leans forward is a positive value, and a forward inclination angle when a user leans backward is a negative value. The forward inclination angle is obtained by converting a pitch angle of the m frame into an angle having such a specification described above. The exercise analysis apparatus 2 (inertial measurement unit 10) may be already inclined, when it is put on a user, and accordingly, the forward inclination angle may be calculated with an amount changed from that point, by assuming that the inclination in the resting state is 0 degrees in a left side view.

### Timing Coincidence

The timing coincidence is an exercise index indicating how close the timings of the feature points of a user are to good timings. For example, an exercise index indicating how close the timing of waist turning is to the timing of the take-off is considered. One leg is still behind the body when the other leg strikes in a running form of a slow leg turnover, and accordingly, when the turning timing of the waist comes after the take-off, this can be determined as the running form of a slow leg turnover. When turning timing of the waist is substantially the same as the timing of take-off, this running form is considered to be good running form. Meanwhile, when the turning timing of the waist is later than the timing of take-off, this running form is considered to be running form with slow leg turnover.

### Leg Turnover

The leg turnover is an exercise index indicating a position of the leg which is behind striking the ground, at the time of the next strike. The leg turnover is, for example, calculated as an angle of the thighbone of the leg behind at the time of strike. For example, an index correlated with the leg turnover can be calculated and then an angle of the thigh bone of the leg behind at the time of strike can be estimated from this index using a predetermined correlation equation.

The index correlated with the leg turnover is, for example, calculated with an expression of (time when the waist is turned toward the thigh bone in the yaw direction−time of strike).

The “time when the waist is turned toward the thigh bone in the yaw direction” is the starting time of the operation of the next step. When the time from the strike to the next operation is long, it can be said that in the time it takes to return the leg that a phenomenon of slow leg turnover occurs.

Alternatively, the index correlated with the leg turnover is, for example, calculated with an expression of (yaw angle when the waist is turned toward the thigh bone in the yaw direction−yaw angle at the time of strike). When a change in the yaw angle from the strike to the next operation is great, an operation of returning the leg after strike is performed and this is shown in the change in the yaw angle. Accordingly, a phenomenon of slow leg turnover occurs.

Alternatively, the pitch angle at the time of strike may be an index correlated with the leg turnover. When the leg is behind at a high position, the body (waist) is inclined forward. Accordingly, the pitch angle of the sensor put on the waist increases. When the pitch angle is large at the time of strike, a phenomenon of slow leg turnover occurs.

1-3-7. Second Analysis Information

Hereinafter, each item of the second analysis information calculated by the second analysis information generation unit 276 will be described in detail.

### Energy Loss

The energy loss is an exercise index indicating an amount of energy wasted relative to the amount of energy consumed for proceeding one step and also indicates a value obtained by integrating an amount of energy wasted relative to the amount of energy consumed for proceeding one step in the running period. The energy loss is calculated using an expression of {exercise energy×(100−direct below strike ratio)×(100−propulsive efficiency)}. Herein, the direct below strike ratio is any one of the direct below strike ratios 1 to 3 and the propulsive efficiency is any one of the propulsive efficiencies 1 to 4.

### Energy Efficiency

The energy efficiency is an exercise index indicating whether the energy consumed for proceeding one step is efficiently used as energy for proceeding in the proceeding direction, and also indicates a value obtained by integrating this in the running period. The energy efficiency is calculated with an expression of {(exercise energy−energy loss)/exercise energy}.

### Strain on Body

The strain on the body is an exercise index indicating the accumulation of the strike impact and a degree of the impact accumulated on the body. Injuries may occur due to the accumulation of the impact, and accordingly, a possibility of occurrence of injuries can also be determined by evaluating the strain on the body. The strain on the body is calculated with an expression of (strain on the right leg+strain on the left leg). The strain on the right leg can be calculated by integrating the strike impact on the right leg. The strain on the left leg can be calculated by integrating the strike impact on the left leg. Herein, both the integration during the running and the integration from the past are performed.

### 1-3-8. Right-Left Difference Ratio (Right and Left Balance)

The right-left difference ratio is an exercise index indicating a degree of a difference between right and left sides of the body regarding the running pitch, the stride, the ground contact time, the impact time, each item of the first analysis information, and each item of the second analysis information, and indicates a degree of non-coincidence of the left leg with respect to the right leg. The right-left difference ratio is calculated with an expression of (numerical value of the left leg/numerical value of the right leg x 100)(%), and the numerical value is each numerical value of the running pitch, the stride, the ground contact time, the impact time, the deceleration amount, the propulsion, the direct below strike ratio, the propulsive efficiency, the speed, the acceleration, the traveling distance, the forward inclination angle, the leg turnover, the angle of waist turning, the angular velocity of waist turning, the amount of inclination to the left and right sides, the running ability, the exercise energy, the energy loss, the energy efficiency, the strike impact, and the strain on the body. The right-left difference ratio also includes an average value of each numerical value and distribution thereof.

### 1-3-9. Procedure of Process

FIG. 17 is a flowchart showing an example of a procedure of an exercise analysis process performed by the processing unit 20. The processing unit 20 executes the exercise analysis process in the order of the flowchart shown in FIG. 17, for example, by executing the exercise analysis program 300 stored in the storage unit 30.

That is, the exercise analysis program 300 is an exercise analysis program which causes a computer to function as the acquisition unit 242 which acquires detection results from a sensor (for example, inertial measurement unit 10) which is attached to a user and detects information relating to the exercise performed by a user, and a right and left determination unit 243 which performs a determination process of determining which period the result indicates of either a first period including a right foot ground contact period or a second period including a left foot ground contact period, based on the acquired detection result.

In the embodiment, an exercise analysis method including: an acquisition step of acquiring the detection result from a sensor (for example, inertial measurement unit 10) which is attached to a user and detects information relating to the exercise performed by a user; and a right and left determination step of performing a determination process of determining which period the result indicates of either a first period including a right foot ground contact period and a second period including a left foot ground contact period, based on the acquired detection result, is realized mainly using the processing unit 20.

As shown in FIG. 17, when the processing unit 20 stands by until the command for starting the measurement is received (N of S10) and receives the command for starting the measurement (Y of S10), the processing unit first assumes that a user stands still, and calculates an initial posture, an initial position, and an initial bias using the sensing data measured by the inertial measurement unit 10 and the GPS data (S20).

The processing unit 20 acquires the sensing data from the inertial measurement unit 10 and adds the acquired sensing data to the sensing data table 310 (S30).

The processing unit 20 performs the inertial navigation operation process and generates the operation data including various information items (S40). An example of the procedure of this inertial navigation operation process will be described later.

The processing unit 20 generates the exercise analysis information by performing the exercise analysis information generation process using the operation data generated in S40 (S50). An example of the procedure of this exercise analysis information generation process will be described later.

The processing unit 20 generates the running output information using the exercise analysis information generated in S50 and transmits the running output information to the notification apparatus 3 (S60).

The processing unit 20 repeats the process in S30 and subsequent processes, every time the sampling period Δt has elapsed (Y of S70) from the acquisition of the previous sampling data, until the command for finishing the measurement is received (N of S70 and N of S80).

When the command for finishing the measurement is received (Y of S80), the processing unit 20 generates the running result information using the exercise analysis information generated in S50, transmits the running result information to the notification apparatus 3 (S90), and finishes the exercise analysis process.

FIG. 18 is a flowchart showing an example of the procedure of the inertial navigation operation process (process in S40 of FIG. 17). The processing unit 20 (inertial navigation operation unit 22) executes the inertial navigation operation process in the order of the flowchart shown in FIG. 18, for example, by executing the inertial navigation operation program 302 stored in the storage unit 30.

As shown in FIG. 18, first, the processing unit 20 removes and corrects the bias from the acceleration and the angular velocity included in the sensing data acquired in S30 of FIG. 17 using the initial bias calculated in S20 of FIG. 17 (using the acceleration bias bₐ and the angular velocity bias b_{ω} after estimating the acceleration bias bₐ and the angular velocity bias b_{ω} in S150 which will be described later), and updates the sensing data table 310 with the corrected acceleration and angular velocity (S100).

The processing unit 20 calculates a speed, a position, and an attitude angle by integrating the sensing data corrected in S100 and adds the calculation data including the calculated speed, position, and attitude angle to the calculation data table 340 (S110).

The processing unit 20 performs a running detection process (S120). An example of the procedure of this running detection process will be described later.

When the running period is detected by performing the running detection process (S120) (Y of S130), the processing unit 20 calculates the running pitch and the stride (S140). When the running period is not detected (N of S130), the processing unit 20 does not perform the process in S140.

The processing unit 20 performs an error estimation process and estimates the speed error δv^{e}, the attitude angle error ε^{e}, the acceleration bias bₐ, the angular velocity bias b_{ω}, and the position error δp^{e} (S150).

The processing unit 20 corrects each of the speed, the position, and the attitude angle using the speed error δv^{e}, the attitude angle error ε^{e}, and the position error εp^{e} estimated in S150 and updates the calculation data table 340 with the corrected speed, position, and attitude angle (S160). The processing unit 20 integrates the speed corrected in S160 and calculates the distance of e frame (S170).

The processing unit 20 performs the coordinate transformation of the sensing data (acceleration and angular velocity of the b frame) stored in the sensing data table 310, the calculation data (speed, position, and attitude angle of the e frame) stored in the calculation data table 340, and the distance of the e frame calculated in S170 into an acceleration, an angular velocity, a speed, a position, an attitude angle, and a distance of the m frame, respectively (S180).

The processing unit 20 generates the operation data including the acceleration, the angular velocity, the speed, the position, the attitude angle, and the distance of the m frame subjected to the coordinate transformation in S180 and the stride, the running pitch, and the right and left foot flag calculated in S140 (S190). The processing unit 20 performs this inertial navigation operation process (process in S100 to S190), each time the sensing data is acquired in S30 of FIG. 17.

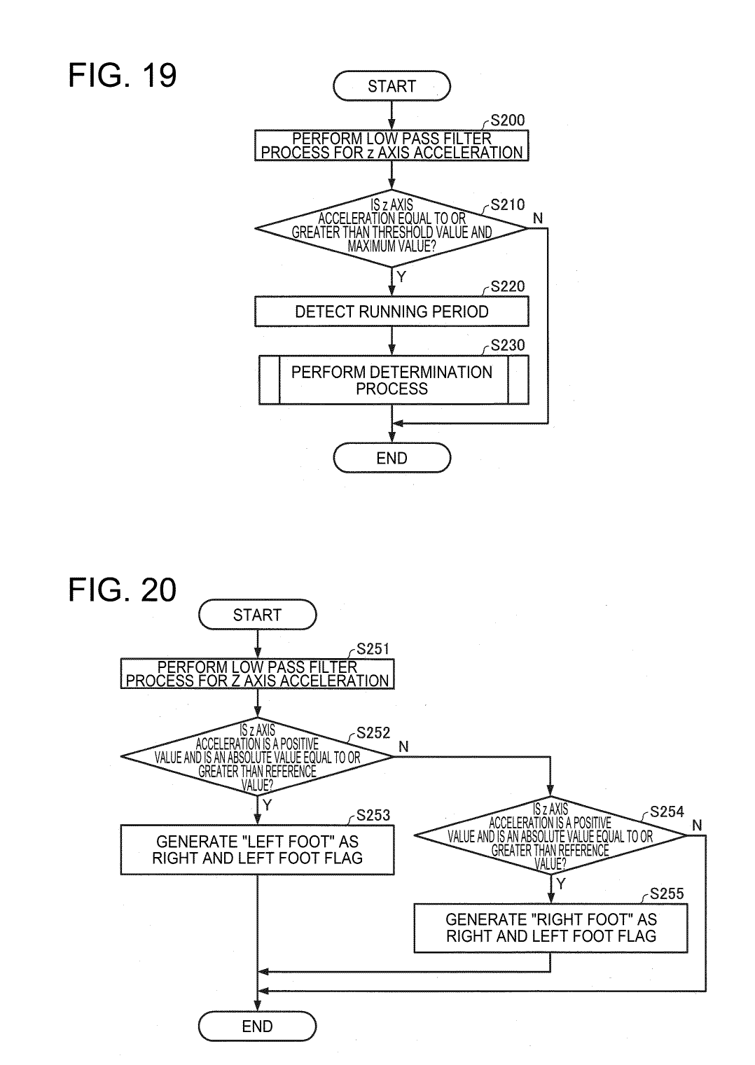

FIG. 19 is a flowchart showing an example of a procedure of a running detection process (process in S120 of FIG. 18). The processing unit 20 (running detection unit 244), for example, executes the running detection process in the order of the flowchart shown in FIG. 19.

As shown in FIG. 19, the processing unit 20 performs a low pass filter process for the z axis acceleration included in the acceleration corrected in S100 of FIG. 18 (S200) and removes noise therefrom.

When the z axis acceleration subjected to the low pass filter process in S200 is equal to or greater than a threshold value or is a maximum value (Y of S210), the processing unit 20 detects the running period at that timing (S220).

The processing unit 20 performs the determination process (S230) and finishes the running detection process. In S230, the processing unit determines whether the running period detected in S220 is the right or left running period and sets the right and left foot flag. When the z axis acceleration is smaller than a threshold value or is not a maximum value (N of S210), the processing unit 20 finishes the running detection process without performing the process in S220 and subsequent processes.

FIG. 20 is a flowchart showing an example of a procedure of the determination process (process in S230 of FIG. 19). The processing unit 20 (right and left determination unit 243), for example, executes the determination process in the order of the flowchart shown in FIG. 20.

As shown in FIG. 20, the processing unit 20 acquires the z axis angular velocity as the detection result from the sensor (acquisition step), performs a low pass filter process of the z axis angular velocity included in the angular velocity corrected in S100 of FIG. 18 (S251), and removes the noise.

When the z axis angular velocity subjected to the low pass filter process in S251 is a positive value and an absolute value thereof is equal to or greater than a reference value (Y of S252), the processing unit 20 generates a flag corresponding to the “left foot” as the right and left foot flag (S253) and finishes the determination process.

When the z axis angular velocity is not a positive value and an absolute value thereof is not equal to or greater than a reference value (N of S252), and the z axis angular velocity subjected to the low pass filter process in S251 is a negative value and an absolute value thereof is equal to or greater than a reference value (Y of S254), the processing unit 20 generates a flag corresponding to the “right foot” as the right and left foot flag (S255) and finishes the determination process.

When the z axis angular velocity is not a negative value and an absolute value thereof is not equal to or greater than a reference value (N of S254), the processing unit finishes the determination process, without updating the right and left foot flag.

FIG. 21 is a flowchart showing an example of a procedure of the exercise analysis information generation process (process in S50 of FIG. 17). The processing unit 20 (exercise analysis unit 24) executes the exercise analysis information generation process in the order of the flowchart shown in FIG. 21, for example, by executing the exercise analysis information generation program 304 stored in the storage unit 30.

As shown in FIG. 21, first, the processing unit 20 calculates each item of the basic information using the operation data generated in the inertial navigation operation process in S40 of FIG. 17 (S300).

The processing unit 20 performs a detection process of the feature points (strike, mid-stance, and the take-off) of the running exercise performed by a user using the operation data (S310).

When the feature points are detected in the process in S310 (Y of S320), the processing unit 20 calculates the ground contact time and the impact time based on the timing when the feature points are detected (S330). The process unit 20 calculates some items of the first analysis information (items to be used with the information regarding the feature points for calculation), based on the timing when the feature points are detected, using a part of the operation data and the ground contact time and the impact time generated in S330 as the input information (S340). When the feature points are not detected in the process of S310 (N of S320), the processing unit 20 does not perform the process in S330 and S340.

The processing unit 20 calculates the other items of the first analysis information (items not to be used with the information regarding the feature points for calculation) using the input information (S350). In S350, the exercise energy of a user is calculated.

The processing unit 20 calculates each item of the second analysis information using the first analysis information (S360).

The processing unit 20 calculates the right-left difference ratio for each item of the input information, each item of the first analysis information, and each item of the second analysis information (S370).

The processing unit 20 adds the current measurement time to each information items calculated in S300 to 370, stores the information items in the storage unit 30 (S380), and finishes the exercise analysis information generation process.

### 1-4. Notification Apparatus

### 1-4-1. Configuration of Notification Apparatus

FIG. 22 is a functional block diagram showing a configuration example of the notification apparatus 3. As shown in FIG. 22, the notification apparatus 3 includes an output unit 110, a processing unit 120, a storage unit 130, a communication unit 140, an operation unit 150, and a time measurement unit 160. However, in the notification apparatus 3, a part of these constituent elements may be removed or changed or other constituent elements may be added.

The storage unit 130 is, for example, configured with a recording medium such as a ROM, a flash ROM, a hard disk, or a memory card which stores a program or data, or a RAM which is a working area of the processing unit 120.

The communication unit 140 is a unit which performs data communication with the communication unit 40 (see FIG. 3) of the exercise analysis apparatus 2, and performs a process of receiving the command (command for starting or finishing the measurement) corresponding to the operation data from the processing unit 120 and transmitting the command to the communication unit 40 of the exercise analysis apparatus 2, a process of receiving the running output information or the running result information which is transmitted from the communication unit 40 of the exercise analysis apparatus 2 and transmitting the information to the processing unit 120, and the like.

The operation unit 150 performs a process of acquiring the operation data from a user (operation data regarding the measurement start and measurement finishing times, operation data regarding selection of display content, and the like) and transmitting the operation data to the processing unit 120. The operation unit 150 may be, for example, a touch panel type display, a button, a key, or a microphone.

The time measurement unit 160 performs a process of generating time information regarding the year, the month, the date, the hour, the minute, the second, and the like. The time measurement unit 160 is realized as a real time clock (RTC) IC, for example.

The output unit 110 outputs the difference information which is generated and output by the output unit 280 of the exercise analysis apparatus 2. The output unit 110 may output the evaluation result which will be described later. In the example shown in FIG. 22, the output unit 110 includes a display unit 170, a sound output unit 180, and a vibration unit 190.

The display unit 170 displays image data or text data transmitted from the processing unit 120 as a letter, a graph, a table, an animation, or other images. The display unit 170 is, for example, realized as a display such as a liquid crystal display (LCD), an organic electroluminescence (EL) display, or an electrophoretic display (EPD), and may be a touch panel type display. The functions of the operation unit 150 and the display unit 170 may be realized with one touch panel type display.

The sound output unit 180 outputs sound data transmitted from the processing unit 120 as sound such as voice or a buzzer. The sound output unit 180 is, for example, realized as a speaker or a buzzer.

The vibration unit 190 vibrates according to vibration data transmitted from the processing unit 120. This vibration is transferred to the notification apparatus 3 and a user equipped with the notification apparatus 3 can feel the vibration. The vibration unit 190 is, for example, realized as a vibration motor or the like.

The processing unit 120 is, for example, configured with a CPU, a DSP, or an ASIC, and performs various operation processes or control processes by executing the programs stored in the storage unit 130 (recording medium). For example, the processing unit 120 performs various processes according to the operation data received from the operation unit 150 (a process of transmitting the command for starting or finishing the measurement to the communication unit 140, a display process or a sound output process according to the operation data), a process of receiving the running output information from the communication unit 140, generating text data or image data corresponding to the exercise analysis information, and transmitting the data to the display unit 170, a process of generating sound data corresponding to the exercise analysis information and transmitting the sound data to the sound output unit 180, and a process of generating vibration data corresponding to the exercise analysis information and transmitting the vibration data to the vibration unit 190. The processing unit 120 performs a process of generating time image data corresponding to time information received from the time measurement unit 160 and transmitting the time image data to the display unit 170.

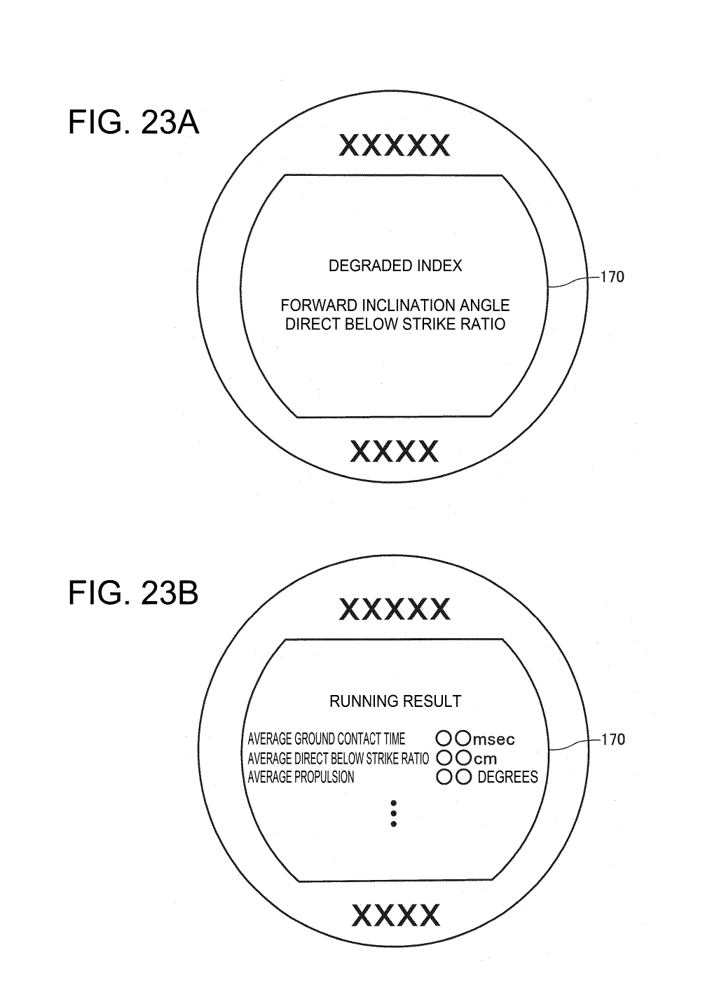

When the right-left difference ratio has an exercise index which is degraded further than a reference value, the processing unit 120 sends notification of the degraded exercise index as sound or vibration and causes the display unit 170 to display the value of the exercise index which is degraded further than the reference value. The processing unit 120 may generate different kinds of sound or vibration depending on the kind of the exercise index which is degraded further than the reference value, or may change the kinds of sound or vibration depending on a degree of degradation further than the reference value for each exercise index. When there are a plurality of exercise indexes which are degraded further than the reference value, the processing unit 120 generates sound or vibration corresponding to the furthest degraded exercise index and may cause the display unit 170 to display the information regarding values of all exercise indexes which are degraded further than the reference value, and the reference value, as shown in FIG. 23A, for example.

The exercise indexes to be compared to the reference value may be all of the exercise indexes included in the running output information, may be only specific exercise indexes which are predetermined, or may be selected by a user operating the operation unit 150.

A user can keep running while grasping the most degraded exercise index and a degree of degradation from the kinds of sound or vibration, without seeing the information displayed on the display unit 170. In addition, when a user sees the information displayed on the display unit 170, a user can properly recognize the difference between values of all of the exercise indexes which are degraded further than the reference value, and the reference value.

The exercise index as a target for causing sound or vibration to be output may be selectable by a user from the exercise indexes to be compared to the reference value, by operating the operation unit 150 or the like. Even in this case, the processing unit may cause the display unit 170 to display the information regarding the values of all exercise indexes which are degraded further than the reference value and the reference value, for example.

A user may perform setting of a notification period (setting of generating sound or vibration for 5 seconds for every minute, for example) through the operation unit 150 and the processing unit 120 may notify a user according to the set notification period.

In the embodiment, the processing unit 120 acquires the running result information transmitted from the exercise analysis apparatus 2 through the communication unit 140 and displays the running result information on the display unit 170. For example, as shown in FIG. 23B, the processing unit 120 displays an average value of each exercise index at the time when running is performed by a user, which is included in the running result information, on the display unit 170. When a user sees the display unit 170 after finishing running (after performing the measurement finishing operation), a user can immediately recognize the suitability of each exercise index.

### 1-4-2. Procedure of Process

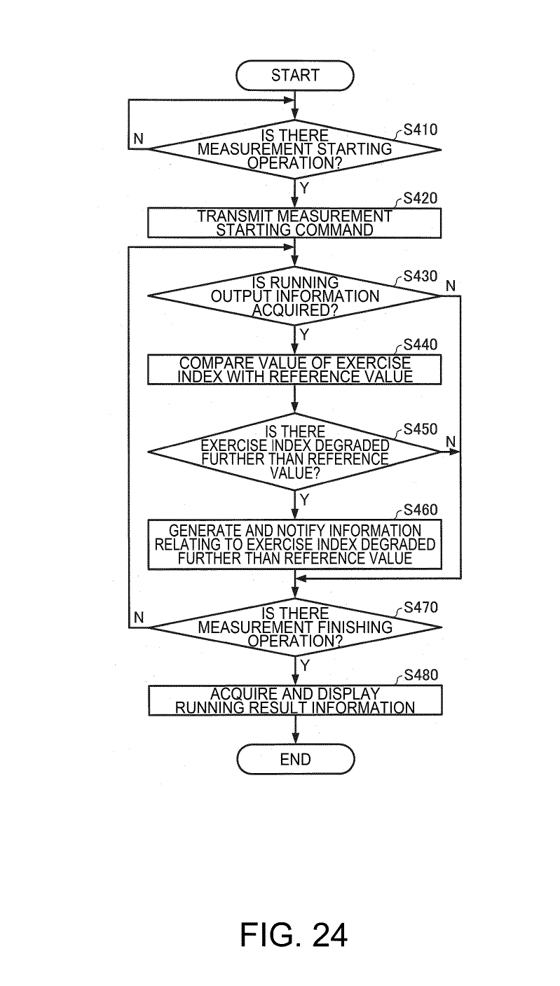

FIG. 24 is a flowchart showing an example of a procedure of a notification process performed by the processing unit 120. The processing unit 120 executes a notification process of the flowchart of FIG. 20, for example, by executing the programs stored in the storage unit 130.

As shown in FIG. 24, first, when the processing unit 120 stands by until the operation data regarding the measurement start is acquired from the operation unit 150 (N of S410) and the operation data regarding the measurement start is acquired (Y of S410), the processing unit 120 transmits the command for starting the measurement to the exercise analysis apparatus 2 through the communication unit 140 (S420).

Until the operation data regarding measurement finishing is acquired from the operation unit 150 (N of S470), the processing unit 120 compares the value of each exercise index included in the acquired running output information with the reference value acquired in S400, each time the running output information is acquired from the exercise analysis apparatus 2 (Y of S430) through the communication unit 140 (S440).

When there is an exercise index which is degraded further than the reference value (Y of S450), the processing unit 120 generates the information regarding the exercise index which is degraded further than the reference value, and notifies a user by sound, vibration, a letter, or the like through the sound output unit 180, the vibration unit 190, and the display unit 170 (S460).

Meanwhile, when there is no exercise index which is degraded further than the reference value (N of S450), the processing unit 120 does not perform a process in S460.

When the operation data regarding the measurement finishing is acquired from the operation unit 150 (Y of S470), the processing unit 120 acquires the running result information from the exercise analysis apparatus 2 through the communication unit 140, causes the display unit 170 to display the running result information (S480), and finishes the notification process.

As described above, a user can run while recognizing the running state, based on the information received in S450. In addition, a user can recognize the running result immediately after finishing the running, based on the information displayed in S480.

### 1-5. Information Analysis Apparatus

1-5-1. configuration of Information Analysis Apparatus

FIG. 25 is a functional block diagram showing a configuration example of the information analysis apparatus 4. As shown in FIG. 25, the information analysis apparatus 4 includes a processing unit 420, a storage unit 430, a communication unit 440, an operation unit 450, a communication unit 460, a display unit 470, and a sound output unit 480. However, in the information analysis apparatus 4, a part of these constituent elements may be removed or changed or other constituent elements may be added.

The communication unit 440 is a unit which performs data communication with the communication unit 40 (see FIG. 3) of the exercise analysis apparatus 2, and performs a process of receiving a transmission requesting command for making a request for transmission of the exercise analysis information designated according to the operation data, from the processing unit 420, transmitting the transmission requesting command to the communication unit 40 of the exercise analysis apparatus 2, receiving the exercise analysis information from the communication unit 40 of the exercise analysis apparatus 2, and transmitting the exercise analysis information to the processing unit 420.

The communication unit 460 is a unit which performs data communication with the server 5, and performs a process of receiving the running data which is a registration target from the processing unit 420, and transmitting the running data to the server 5 (registration process of running data), a process of receiving management information corresponding to the operation data such as editing, removing, or replacing of the running data from the processing unit 420 and transmitting the management information to the server 5, and the like.

The operation unit 450 performs a process of acquiring the operation data the operation data such as (registration of running data, editing, removing, or replacing) from a user and transmitting the operation data to the processing unit 420. The operation unit 450 may be, for example, a touch panel type display, a button, a key, or a microphone.

The display unit 470 displays image data or text data transmitted from the processing unit 420 as a letter, a graph, a table, an animation, or other images. The display unit 470 is, for example, realized as a display such as an LCD, an organic EL display, or an EPD, and may be a touch panel type display. The functions of the operation unit 450 and the display unit 470 may be realized with one touch panel type display.

The sound output unit 480 outputs sound data transmitted from the processing unit 420 as sound such as voice or a buzzer. The sound output unit 480 is, for example, realized as a speaker or a buzzer.

The storage unit 430 is, for example, configured with a recording medium such as a ROM, a flash ROM, a hard disk, or a memory card which stores a program or data, or a RAM which is a working area of the processing unit 420. An analysis program 432 which is to be read out by the processing unit 420 for executing an analysis process is stored in the storage unit 430 (any recording medium).

The processing unit 420 is, for example, configured with a CPU, a DSP, or an ASIC, and performs various operation processes or control processes by executing the various programs stored in the storage unit 430 (recording medium). For example, the processing unit 420 performs a process of transmitting a transmission requesting command for making a request for transmission of the exercise analysis information designated according to the operation data received from the operation unit 450 to the exercise analysis apparatus 2 through the communication unit 440 and receiving the exercise analysis information from the exercise analysis apparatus 2 through the communication unit 440, or a process of generating running data including the exercise analysis information received from the exercise analysis apparatus 2 according to the operation data received from the operation unit 450 and transmitting the running data to the server 5 through the communication unit 460. The processing unit 420 performs a process of transmitting management information corresponding to the operation data received from the operation unit 450 to the server 5 through the communication unit 460. The processing unit 420 performs a process of transmitting a transmission request for the running data which is an evaluation target selected according to the operation data received from the operation unit 450 to the server 5 through the communication unit 460, and receiving the running data which is the evaluation target from the server 5 through the communication unit 460. The processing unit 420 performs a process of analyzing the running data which is an evaluation target selected according to the operation data received from the operation unit 450, generating analysis information which is information regarding the analysis result, and transmitting the analysis information as text data, image data, or sound data, to the display unit 470 or the sound output unit 480.

In the embodiment, the processing unit 420 functions as an exercise analysis information acquisition unit 422 and an analysis information generation unit 424 by executing the analysis program 432 stored in the storage unit 430. However, the processing unit 420 may receive and execute the analysis program 432 stored in an arbitrary storage apparatus (recording medium) through a network.

The exercise analysis information acquisition unit 422 performs a process of acquiring the exercise analysis information which is information regarding the analysis result of the exercise performed by a user who is an analysis target, from database of the server 5 (or exercise analysis apparatus 2). The exercise analysis information acquired by the exercise analysis information acquisition unit 422 is stored in the storage unit 430. The exercise analysis information may be generated by the same exercise analysis apparatus 2 or may be generated by any of a plurality of different exercise analysis apparatuses 2. The plurality of items of the exercise analysis information acquired by the exercise analysis information acquisition unit 422 may include values of various exercise indexes (for example, various exercise indexes regarding the right and left foot described above) of a user.

The analysis information generation unit 424 performs a process of generating the analysis information relating to the running ability of a user who is an analysis target, using the exercise analysis information acquired by the exercise analysis information acquisition unit 422. The analysis information generation unit 424, for example, may generate the analysis information using the exercise analysis information of a user who is an analysis target in the period selected in the operation data acquired from the operation unit 450.

The analysis information generation unit 424 may generate the analysis information for comparing the running ability for each date when a user who is an analysis target performed the running. For example, when a user performed the running on the 1^{st} of July, the 8^{th} of July, and the 15^{th} of July, the analysis information generation unit may generate the analysis information for comparing the running ability of a user for each date including the 1^{st} of July, the 8^{th} of July, and the 15^{th} of July.

The analysis information generation unit 424 may generate the analysis information for evaluating the running ability of a user who is an analysis target, using a value of the exercise index which is registered in the database of the server 5 and included in each of the running data item (exercise analysis information item) of a plurality of users relative to a user who is an analysis target. For example, the plurality of users may be users who are selected from the operation data received from the operation unit 450. For example, the highest index value is set as 10 and the lowest index value is set as 0 among the exercise index values of the plurality of users, and the analysis information generation unit 424 may convert the exercise index values of a user who is an analysis target into values of 0 to 10 and generate the analysis information including information regarding the converted exercise index values, and may calculate a deviation of the exercise index value of a user who is an analysis target using the exercise index values of the plurality of users and generate the analysis information including information regarding the deviation.

The processing unit 420 generates display data such as text or an image or sound data such as voice using the analysis information generated by the analysis information generation unit 424 and outputs the data to the display unit 470 or the sound output unit 480. Accordingly, the analysis result of a plurality of users who are analysis targets is presented by the display unit 470 or the sound output unit 480.

### 1-5-2. Procedure of Process

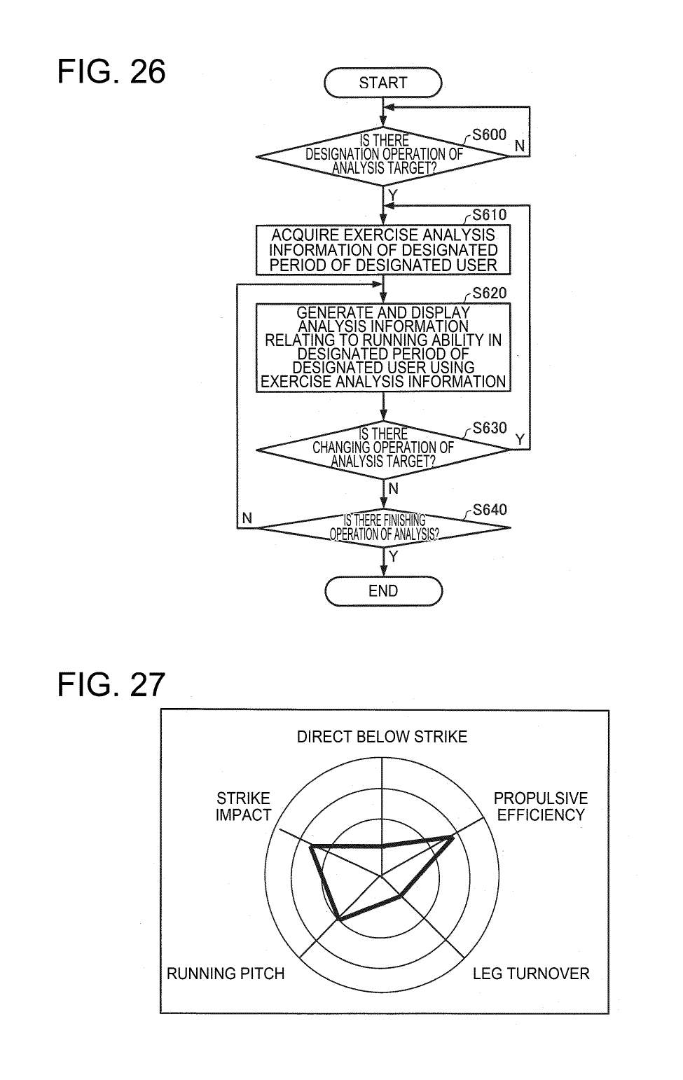

FIG. 26 is a flowchart showing an example of a procedure of an analysis process performed by the processing unit 420. The processing unit 420 executes an analysis process in the order of the flowchart shown in FIG. 26, for example, by executing the analysis program 432 stored in the storage unit 430.

First, when the processing unit 420 stands by until the operation data designating the analysis target is acquired (N of S600) and the operation data designating the analysis target is acquired (Y of S600), the processing unit 420 acquires the exercise analysis information (specifically, running data) regarding the designated period (period which is an analysis target) of the designated user (user who is an analysis target) in the operation data from the database of the server 5 through the communication unit 460, and stores the exercise analysis information in the storage unit 430 (S610).

The processing unit 420 generates the analysis information regarding the running ability in the designated period (period which is an analysis target) of the designated user (user who is an analysis target), using the exercise analysis information (running data) acquired in S610, and displays the analysis information on the display unit 270 (S620).

When the operation data for changing the analysis target or the operation data for finishing the analysis is not acquired (N of S630 and N of S640), the processing unit 420 performs the process in S620.

When the operation data for changing the analysis target is acquired (Y of S630), the processing unit 420 performs the processes in S610 and S620 again, and when the operation data for finishing the analysis is acquired (Y of S640), the processing unit finishes the analysis process.

FIG. 27 is a diagram showing an example of the analysis information displayed on the display unit 270. In an example shown in FIG. 27, the analysis information displayed on the display unit 270 includes a radar chart in which right-left difference ratios of the five exercise indexes (the direct below strike, the propulsive efficiency, the leg turnover, the running pitch, and the strike impact) of the running in the period which is an analysis target of a user who is an analysis target, are evaluated by being compared with an average value of a plurality of selected users. For example, when a user selects a plurality of users while setting himself or herself as a user who is an analysis target and selects the period which is an analysis target through the operation unit 450, the processing unit 420 acquires the exercise analysis information (value of each exercise index) regarding all running operations executed in the selected period and performed by the plurality of selected users from the database of the server 5. The processing unit 420 calculates an average value of exercise indexes of the users, converts the value of the user who is an analysis target into the relatively evaluated value, by setting the highest value among the exercise indexes of the plurality of users as 10 and the lowest value as 0, and generates the radar chart shown in FIG. 27. A user can evaluate his or her running ability relative to the plurality of selected users (for example, member of a running team), using the analysis information shown in FIG. 27. For example, in the example shown in FIG. 27, it is found that the “leg turnover” and the “direct below strike” are relatively low (weak points), and accordingly, a user is conscious of the need for improvement of the exercise indexes, and therefore, improvement of the running result may be a target the next time a user runs.

### 1-6. Effects

According to the embodiment, the determination process can be performed with one sensor, and it is possible to realize the exercise analysis apparatus 2 and the exercise analysis system 1 which can grasp exercise motions of right and left foot with a simple configuration.

Particularly, since the inertial sensor (inertial measurement unit 10) can detect small movements of a user equipped with the sensor, it is possible to realize the exercise analysis apparatus 2 and the exercise analysis system 1 which can precisely grasp exercise motions of right and left foot with one sensor.

In a walking operation or a running operation, a rotation exercise in the yaw direction occurs in the body of a user. Therefore, according to the embodiment, it is possible to realize the exercise analysis apparatus 2 and the exercise analysis system 1 which can precisely grasp exercise motions of right and left foot with one sensor.

In a walking operation or a running operation, there is a direction orthogonal to both the proceeding direction of the user and the vertical direction. Therefore, according to the embodiment, it is possible to realize the exercise analysis apparatus which can precisely grasp exercise motions of right and left foot with one sensor.

According to the embodiment, the determination process can be performed based on the running timing (for example, timing of one step) and the detection result of the sensor, and therefore, it is possible to realize the exercise analysis apparatus 2 and the exercise analysis system 1 which can precisely grasp exercise motions of right and left foot.

According to the embodiment, it is possible to realize the exercise analysis apparatus 2 and the exercise analysis system 1 which can provide a user with the information relating to the right and left balance of the exercise performed by a user.

According to the embodiment, it is possible to realize the exercise analysis apparatus 2 and the exercise analysis system 1 which can provide the information relating to the right and left balance of the exercise performed by a user which a user can easily understand.

According to the embodiment, it is possible to realize the exercise analysis apparatus 2 and the exercise analysis system 1 which can send a notification to a user, when the right and left balance of the exercise performed by a user is not obtained.

### 2. Modification Examples

The invention is not limited to the embodiment and various modifications can be performed within a range of a gist of the invention. Hereinafter, modification examples will be described. The same reference numerals are used for the same configuration elements as those of the embodiment described above and the overlapping description will be omitted.

2-1. Sensor

In the embodiment, the acceleration sensor 12 and the angular velocity sensor 14 are integrally embedded in the exercise analysis apparatus 2 as the inertial measurement unit 10, but the acceleration sensor 12 and the angular velocity sensor 14 may not be integrated. Alternatively, the acceleration sensor 12 and the angular velocity sensor 14 may not be embedded in the exercise analysis apparatus 2 and may be directly put on a user. In both cases, a coordinate system of any one of the sensors may be converted into the b frame of the embodiment and a coordinate system of the other sensor may be converted into the b frame, and the embodiment described above may be applied thereto.

In the embodiment described above, a part of a user for attaching the sensor (exercise analysis apparatus 2 (IMU 10)) is described as the waist, but the sensor may be put on a part other than the waist. A preferred part for attaching the sensor is a trunk of the body of a user (part other than arms and legs). However, the preferred part is not limited to the trunk of the body, and the sensor may be put on the head or a leg of a user, for example, rather than an arm. In addition, the number of sensors is not limited to one and an additional sensor may be put on another part of the body. For example, the sensors may be put on the waist and the leg, or the waist and the arm.

### 2-2. Inertial Navigation Operation

In the embodiment described above, the integration processing unit 220 calculates the speed, the position, the attitude angle, and the distance of the e frame and the coordinate transformation unit 250 performs the coordinate transformation for those of the e frame into the speed, the position, the attitude angle, and the distance of the m frame, but the integration processing unit 220 may calculate the speed, the position, the attitude angle, and the distance of the m frame. In this case, the exercise analysis unit 24 may perform the exercise analysis process using the speed, the position, the attitude angle, and the distance of the m frame calculated by the integration processing unit 220, and accordingly, the coordinate transformation of the speed, the position, the attitude angle, and the distance by the coordinate transformation unit 250 is unnecessary. In addition, the error estimation unit 230 may perform error estimation performed with the extended Karman filter using the speed, the position, and the attitude angle of the m frame.

In the embodiment, the inertial navigation operation unit 22 performs a part of the inertial navigation operation using the signal from the GPS satellite, but may use a signal from a satellite for positioning of a global navigation satellite system (GNSS) rather than the GPS or a satellite for positioning rather than the GNSS. One or two or more satellite positioning systems such as the Wide Area Augmentation System (WAAS), the Quasi Zenith Satellite System (QZSS), the Global Navigation Satellite System (GLONASS), GALILEO, and the BeiDou Navigation Satellite System (BeiDou) may be used. The Indoor Messaging System (IMES) may also be used.

In the embodiment described above, the running detection unit 244 detects the running period at the timing when the acceleration (z axis acceleration) of a vertical motion of a user is equal to or greater than a predetermined threshold value and is a maximum value, but there is no limitation thereon, and the running detection unit may detect the running period at the timing when the acceleration (z axis acceleration) of a vertical motion is changed from a positive value to a negative value (or the timing which is changed from a negative value to a positive value). Alternatively, the running detection unit 244 may calculate the speed (z axis speed) of a vertical motion by integrating the acceleration (z axis acceleration) of a vertical motion and detecting the running period using the calculated speed (z axis speed) of a vertical motion. In this case, the running detection unit 244 may detect the running period at the timing when the speed crosses the threshold value which is close to a median of the maximum value and the minimum value due to an increase or a decrease of the value. For example, the running detection unit 244 may calculate a resultant acceleration of the x axis, the y axis, and the z axis and detect the running period using the calculated resultant acceleration. In this case, the running detection unit 244 may detect the running period at the timing when the resultant acceleration crosses the threshold value which is close to a median of the maximum value and the minimum value due to an increase or a decrease of the value.

In the embodiment described above, the error estimation unit 230 sets the speed, the attitude angle, the acceleration, the angular velocity, and the position as state variables and estimates the errors thereof using the extended Karman filter, but may set a part of the speed, the attitude angle, the acceleration, the angular velocity, and the position as state variables and estimate the errors thereof. Alternatively, the error estimation unit 230 may set elements (for example, traveling distance) other than the speed, the attitude angle, the acceleration, the angular velocity, and the position as state variables and estimate the errors thereof.

In the embodiment described above, the extended Karman filter is used in the estimation of the errors performed by the error estimation unit 230, but other estimation units such as a particle filter or H co (H infinity) filter may be substituted.

### 2-3. Exercise Analysis Process

In the embodiment described above, the exercise analysis apparatus 2 performs the generation process of the exercise analysis information (exercise indexes), but the exercise analysis apparatus 2 may transmit the measurement data of the inertial measurement unit 10 or the operation results (operation data) of the inertial navigation operation to the server 5, and the server 5 may perform the generation process of the exercise analysis information (exercise indexes) (may function as the exercise analysis apparatus), using the measurement data or the operation data, and store the exercise analysis information in the database.

For example, the exercise analysis apparatus 2 may generate the exercise analysis information (exercise indexes) using biological information of a user. As the biological information, cutaneous temperature, core temperature, oxygen consumption, a variation in heart beat, a heart rate, a pulse rate, a respiratory rate, a heat flow, galvanic skin response, an electromyogram (EMG), an electroencephalogram (EEG), an electro-oculogram (EOG), blood pressure, another activity, and the like are considered, for example. The exercise analysis apparatus 2 may include a device which measures the biological information or the exercise analysis apparatus 2 may receive the biological information measured through a measurement device. For example, a user may be equipped with a watch type pulsimeter or may run with a heart rate sensor strapped on the chest by a belt, and the exercise analysis apparatus 2 may calculate the heart rate of a user during running, using a measured value of the pulsimeter or the heat rate sensor.

In the embodiment, each exercise index included in the exercise analysis information is an index relating to technique of a user, but the exercise analysis information may also include the exercise index relating to endurance. For example, the exercise analysis information may include heart rate reserve (HRR) calculated with an expression of (heart rate−heart rate during rest)/(maximum heart rate−heart rate during rest)×100, as the exercise index relating to endurance. For example, the notification apparatus 3 may be operated for inputting the heart rate, the maximum heart rate, and the heart rate during rest, every time each user runs, or a user may run while being equipped with a heart rate meter, and the exercise analysis apparatus 2 may acquire values of the heart rate, the maximum heart rate, and the heart rate during rest from the notification apparatus 3 or the heart rate meter, and calculate a value of the heart rate reserve (HRR).

In the embodiment described above, the exercise analysis of the running by a person is a target, but there is no limitation thereto, and the invention can also be applied to the exercise analysis of walking or running of an animal or a moving body such as a walking robot. The exercise is not limited to running, and the invention can also be applied to various exercises such as mountain climbing, trail running, skiing (including cross-country skiing or ski jumping), snowboarding, swimming, cycling, skating, golf, tennis, baseball, and rehabilitation. In the case of being applied to skiing, as an example, good carving performance or shifting of skis may be determined from a variation in the acceleration in the vertical direction at the time of applying pressure against the skis, or a difference between the right and left foot or sliding ability may be determined from tracking of a change in the acceleration in the vertical direction at the time of applying pressure against and unloading pressure applied to the skis. Alternatively, whether or not a user wears skis may be determined by analyzing similarity between tracking of a change in the angular velocity in the yaw direction and the sine wave, or the smooth sliding performance may be determined by analyzing similarity between tracking of a change in the angular velocity in the roll direction and the sine wave.

### 2-4. Notification Process

In the embodiment described above, the notification apparatus 3 notifies a user with sound or vibration, when there are exercise indexes which are degraded further than the reference value, however, the notification apparatus 3 may notify a user with sound or vibration, when there are exercise indexes which are improved further than the reference value.

In the embodiment described above, the notification apparatus 3 performs the comparing process between the value of each exercise index and the reference value, but the exercise analysis apparatus 2 may perform this comparing process and control output of sound or vibration or display performed by the notification apparatus 3 according to the compared results.

In the embodiment described above, the notification apparatus 3 is a watch type apparatus, but there is no limitation thereto, and the notification apparatus may be a portable apparatus other than the watch type to be put on a user (head mounted display (HMD) or a device put on the waist of a user (may be the exercise analysis apparatus 2)) or a portable device which is not wearable (smart phone). When the notification apparatus 3 is a head mount display (HMD), a display unit thereof has sufficiently excellent visibility compared to that of the display unit of the watch type notification apparatus 3, and accordingly, when a user sees the display unit, this does not disturb the running Therefore, information regarding running transition of a user to the current state, or a moving image showing running of a virtual runner created based on the time (time set by a user, recording personal record, the record of a celebrity, a world record, or the like) may be displayed.

### 2-5. Analysis Process

In the embodiment described above, the information analysis apparatus 4 performs the analysis process, but the server 5 may perform the analysis process (may function as the information analysis apparatus) and the server 5 may transmit the analysis information to a display apparatus through a network.

In the embodiment described above, the running data (exercise analysis information) of a user is stored in the database of the server 5, but may be stored in a database created in the storage unit 430 of the information analysis apparatus 4. That is, the server 5 may not be provided.

### 2-6. Others

For example, the exercise analysis apparatus 2 or the notification apparatus 3 may calculate points of a user from the input information or the analysis information and may notify the points during running or after running. For example, the numerical values of the exercise indexes may be divided into a plurality of steps (for example, 5 steps or 10 steps) and points may be set for each step. For example, the exercise analysis apparatus 2 or the notification apparatus 3 may apply points according to the type or the number of exercise indexes having a good record or calculate the total points, and display the result.

In the embodiment described above, the GPS unit 50 is provided in the exercise analysis apparatus 2, but may be provided in the notification apparatus 3. In this case, the processing unit 120 of the notification apparatus 3 may receive the GPS data from the GPS unit 50 and transmit the GPS data to the exercise analysis apparatus 2 through the communication unit 140, the processing unit 20 of the exercise analysis apparatus 2 may receive the GPS data through the communication unit 40, and add the received GPS data to the GPS data table 320.

In the embodiment described above, the exercise analysis apparatus 2 and the notification apparatus 3 are separately provided, however, the exercise analysis apparatus including the integrated exercise analysis apparatus 2 and the notification apparatus 3 may be provided.

In the embodiment described above, the exercise analysis apparatus 2 is put on a user, but there is no limitation thereto, and the inertial measurement unit (inertial sensor) or the GPS unit may be put on the body of a user, the inertial measurement unit (inertial sensor) or the GPS unit may transmit each detection result to a portable information apparatus such as a smart phone or a stationary information apparatus such as a personal computer, or the server through a network, and these apparatuses may analyze the exercise of a user using the received detection result. Alternatively, the inertial measurement unit (inertial sensor) or the GPS unit put on the body of a user may record the detection result in a recording medium such as a memory card, the information apparatus such as a smart phone or a personal computer may read out the detection result from the recording medium and perform the exercise analysis process.

The embodiments and modification examples described above are merely an example and the invention is not limited thereto. Each embodiment and each modification example can be suitably combined with each other, for example.

The invention includes the substantially the same configuration (for example, the configuration with the same function, method, and result, or the configuration with the same object and effect) as the configuration described in the embodiments. The invention includes the configuration obtained by replacing the non-substantial part of the configuration described in the embodiments. The invention includes the configuration which realizes the same action effect as the configuration described in the embodiments or the configuration which can achieve the same object. The invention includes the configuration obtained by adding a well-known technology to the configuration described in the embodiments.

The entire disclosure of Japanese Patent Application No. 2014-157205, filed Jul. 31, 2014 is expressly incorporated by reference herein.

What is claimed is:

1. An exercise analysis apparatus comprising:
an acquisition unit which acquires a detection result from a sensor which detects information relating to an exercise performed by a user; and
a right and left determination unit which performs a determination process of determining whether a period is a first period including a right foot ground contact period of the user or a second period including a left foot ground contact period of the user, based on the detection result.

2. The exercise analysis apparatus according to claim 1,
wherein the acquisition unit acquires at least one information item regarding an angle and an angular velocity.

3. The exercise analysis apparatus according to claim 1,
wherein the right and left determination unit performs the determination process based on an angular velocity of the user in a yaw direction.

4. The exercise analysis apparatus according to claim 1,
wherein the right and left determination unit performs the determination process based on an acceleration in a direction orthogonal to both of a proceeding direction of the user and a vertical direction.

5. The exercise analysis apparatus according to claim 1,
wherein the right and left determination unit performs the determination process based on an angle of the user in the yaw direction.

6. The exercise analysis apparatus according to claim 1, further comprising:
a running detection unit which detects running timing of the user,
wherein the right and left determination unit performs the determination process based on the running timing.

7. The exercise analysis apparatus according to claim 1, further comprising:
a calculation unit which calculates the index relating to the exercise performed by the user,
wherein the calculation unit calculates the index in the first period and the index in the second period.

8. The exercise analysis apparatus according to claim 7,
wherein the calculation unit calculates a difference between the index in the first period and the index in the second period.

9. The exercise analysis apparatus according to claim 8,
wherein the index is at least one of direct below strike, propulsive efficiency, leg turnover, a running pitch, and strike impact.

10. The exercise analysis apparatus according to claim 8, further comprising:
an output unit which outputs difference information which is information relating to the difference, when the difference is equal to or greater than a reference value.

11. An exercise analysis system comprising:
the exercise analysis apparatus according to claim 10; and
a notification apparatus which outputs the difference information output by the exercise analysis apparatus.

12. An exercise analysis method comprising:
acquiring a detection result from a sensor which detects information relating to an exercise performed by a user; and
performing a determination process of determining whether a period is a first period including a right foot ground contact period of the user or a second period including a left foot ground contact period of the user, based on the detection result.

13. An exercise analysis program which causes a computer to function as:
an acquisition unit which acquires a detection result from a sensor which detects information relating to an exercise performed by a user; and
a right and left determination unit which performs a determination process of determining whether a period is a first period including a right foot ground contact period of the user or a second period including a left foot ground contact period of the user, based on the detection result.
